(19) United States
(12) Patent Application Publication
(10) Pub. No.: US 2010/0284925 A1
(43) Pub. Date: Nov. 11, 2010

(54) Small Molecule Intervention for Obesity

(76) Inventor: Khew-Voon Chin, Toledo, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/445,815

(22) PCT Filed: Oct. 17, 2007
(86) PCT No.: PCT/US07/22144

§ 371 Date: Apr. 16, 2009

Related U.S. Application Data
(60) Provisional application No. 60/852,358, filed on Oct. 17, 2006.

(51) Int. Cl. A61K 49/00; C07D 493/22; A61K 31/352; A61P 3/04

(52) U.S. Cl. 424/9.2; 549/275; 514/453

(57) ABSTRACT

Methods and compositions for activating PLTP gene expression include administering an effective amount of a limonoid.

### CROSS-REFERENCE TO RELATED APPLICATIONS

This application claims the benefit of PCT application No. PCT/US07/22144 filed Oct. 17, 2007 which claims priority to U.S. Provisional Application No. 60/852,358, filed Oct. 17, 2006, the disclosures of which are incorporated herein by reference.

### TECHNICAL FIELD AND INDUSTRIAL APPLICABILITY OF THE INVENTION

Prieurianin is a novel anti-obesity drug that targets adipogenesis. Prieurianin inhibits the proliferation and differentiation of preadipocytes, as well as reduces the number of lipid positive adipocytes in differentiated culture. Also, prieurianin is an important pharmacological tool for probing the biochemistry and physiology of adipogenesis.

### BACKGROUND OF THE INVENTION

The increase in incidence of obesity and its associated health problems have had a significant impact on the cost of global health care in recent years. In the United States alone, it is estimated that approximately two-thirds of the adults are overweight, with one third of these considered obese. The alarming rate of increase in obesity is largely due to a sedentary lifestyle habits coupled with overconsumption of energy-rich foods, which create a chronic energy imbalance that leads to weight gain in the form of body fat. As adiposity increases, the risk of developing comorbidities such as diabetes, hypertension, and cardiovascular disease is also significantly elevated. It is also recognized that not all fats are created equal, but that the accumulation of visceral adipose tissue, not subcutaneous fat, increases the risk of cardiovascular and metabolic diseases. In fact, obesity is a major factor in triggering the onset of insulin resistance, dyslipidemia (characterized by hypertriglyceridemia), low levels of high density lipoproteins cholesterol (HDL-C), small, dense HDL particles and increased phospholipid transfer protein (PLTP) activity. Hence, this increased prevalence of obesity and the whole host of its comorbidities worldwide warrant urgent effective therapeutic drugs and alteration of life style inventions to combat an emerging health problem that threatens billions of people globally.

The discovery of leptin and its weight-reducing pharmacological effects more than a decade ago has led to new understanding of adipose tissue function. Adipose tissue is now known to not only store and release fatty acids, but also to produce a number of hormonal factors or adipokines that have tremendous impact on the regulation of body weight and homeostasis of blood glucose. Adipose tissue acts as an endocrine organ and produces a number of substances with an important role in the regulation of food intake, energy expenditure and a series of metabolic processes. Also, adipocytes express and release proteins that are engaged in signaling pathways as well as playing critical roles in energy storage and metabolism.

These advances further defined that the white adipose tissue actually plays a central role in the regulation of energy balance and acts as a secretory/endocrine organ that mediates numerous physiological and pathological processes. Dysregulation of white adipose tissue mass causes obesity or lipoatrophy. Alterations in white adipose tissue mass resulting from changes in adipocyte size and/or number, are regulated by a complex interplay between proliferation and differentiation of preadipocytes, and the various proteins and factors secreted by adipocytes.

One of the major hormonal factors released by adipose tissue is adiponectin, a recently discovered hormone produced exclusively by adipocytes. Adiponectin is abundantly present in the plasma and has been shown to increase insulin sensitivity by stimulating fatty acid oxidation, decrease plasma triglycerides and improve glucose metabolism. Adiponectin levels are inversely related to the degree of adiposity and its level is significantly reduced in obese subjects. Clinically, decreased adiponectin level in the plasma is also associated with obesity-related insulin resistance and atherosclerosis. The anti-atherogenic and anti-inflammatory properties of adiponectin and its ability to stimulate insulin sensitivity have made adiponectin an important target for physiological and pathophysiological studies with the aim of potential therapeutic applications.

In addition to adiponectin, other proteins including resistin, visfatin, tumor necrosis factor α, and acylation-stimulating protein, constitute a diverse array of adipocyte-derived hormones and cytokines that serve to orchestrate the response of adipose tissue to both central and peripheral metabolic signals. Some of these proteins also have been validated as candidate drug targets for the development of therapeutics to treat obesity, and are now in drug development stages. These advances provide hope that the current obesity epidemic can be effectively treated with drugs in the near future.

Phospholipid Transfer Protein (PLTP) in Obesity—Dyslipidemia associated with obesity is marked by hypertriglyceridemia, low HDLC, and increased plasma PLTP activity. In human, it is now believed that plasma PLTP activity is significantly elevated in obese subjects, as well as in insulin resistance and type 2 diabetes mellitus in association with high plasma triglycerides and obesity. Also, pronounced weight loss after gastric banding surgery resulted in a significant decrease of PLTP activity. PLTP is thought to function in reverse cholesterol transport in regulating the size and composition of HDL and hence controlling plasma HDL levels. Hence, the paradoxical increase in plasma PLTP activity in obese individuals raised questions as to what role does it play in obesity.

Profile of PLTP—Human PLTP is a major serum protein encoded by a gene containing 16 exons, spanning approximately 13 kb on chromosome 20q12-q13.1, with a cDNA of 1750 base pairs and 476 amino acids long.

The molecular weight of purified PLTP on SDS-PAGE is approximately 81 kDa, much larger than the protein mass predicted from the cDNA and its mRNA transcript is found in a variety of tissues including pancreas, lung, kidney, heart, liver, skeletal muscle and brain, as well as in the adipose tissues exhibiting a depot-related difference between subcutaneous and visceral adipose tissues. PLTP is a member of the lipopolysaccharide-binding/lipid transfer protein family, which includes the cholesteryl ester transfer protein, lipopolysaccharide-binding protein and bactericidal/permeability-increasing protein. The crystal structure of the bacterial/permeability increasing protein reveals that proteins in this family (including PLTP) contain intrinsic lipid binding sites and appear to act as carrier proteins that shuttle between lipoproteins to redistribute lipids.

The predicted model structure of PLTP consists of two lipid-binding pockets characterized by apolar residues, with an N-terminal pocket critical for PLTP transfer activity and a C-terminal pocket involved in lipid binding.

Function of PLTP—Proatherogenic or Antiatherogenic—PLTP shuttles excess surface phospholipids and cholesterol from triglyceride-rich lipoproteins to HDL in reverse cholesterol transport during intravascular lipolysis of chylomicrons and VLDL. Further, in vitro studies showed that PLTP transfers different phospholipids and free cholesterol between lipoproteins and reconstituted vesicles. PLTP is also capable of modifying HDL particle size distribution, a process called HDL conversion or remodeling that results in the formation of pre-β-HDL, which is thought to be an efficient acceptor of cholesterol. In addition, PLTP deficiency in mice by homologous recombination knockout provides an approximately 50% reduction in HDL levels, thus indicating its essential role in transferring phospholipids from triglyceride rich lipoproteins into HDL. Intriguingly, overexpression of PLTP also lowers plasma HDL levels. It is now believed that there is an antiatherogenic potential of PLTP, while others have found plasma PLTP level and activity to be positively and independently correlated with coronary artery disease, and that transgenic mouse models with increased susceptibility for the development of atherosclerosis, bred into either PLTP knockout or overexpressing mice, demonstrated the proatherogenic role of PLTP.

In addition, PLTP mRNA levels and activity are consistently associated with obesity, thus suggesting that PLTP might have another function in the regulation of body fat. This functional significance between PLTP and obesity is not fully understood, but it has been speculated that increased synthesis of PLTP may be a result of the enlarged mass of adipose tissue, as PLTP activity is decreased following weight loss. These results seem to be consistent with genome-wide scans studies that showed significant evidence of linkage with obesity-related phenotypes within the chromosomal locus of PLTP. Moreover, in several mouse studies genes influencing body fatness residing on chromosome 2 are syntenic with a region on human chromosome 20q.

Low PLTP has also been shown to be directly related to increased waist circumference. In addition, paradoxically, it was shown in Caenorhabditis elegans that inactivation of the PLTP gene by RNA interference causes an increase in fat storage, thus suggesting that functional mutations in the mammalian PLTP homolog could lead to obesity.

The inventor herein, in Chin U.S. Pat. No. 7,078,411, identified a process for increasing reverse cholesterol transport by administering camptothecin or a camptothecin derivative to promote the increase expression of PLTP. The inventor has now provided herein a further advance that is useful to regulate PLTP in an effective manner.

There is also provided herein an advance that is useful to reduce the incidence of obesity and its related health related problems.

In addition, there is also provided herein an advance that is useful in the development of effective therapeutic drugs to regulate an individual's body weight.

### SUMMARY OF THE INVENTION

In one aspect, the present invention relates to a method for transcriptionally activating phospholipid transfer protein (PLTP) gene expression by administering an effective amount of a limonoid such as prieurianin.

In another aspect, the present invention relates to a method to induce a significant weight loss and/or a reduction in food intake by administering an effective amount of prieurianin.

In yet other aspect, the present invention further provides the following:

a method to decrease visceral and subcutaneous adipose tissues comprising administering an effective amount of prieurianin;

a method to decrease the serum non-esterified fatty acid levels comprising administering an effective amount of prieurianin;

a method to inhibit the proliferation and differentiation of preadipocytes comprising administering an effective amount of prieurianin; and

a method to cause either de-differentiation or a loss of fat accumulation in adipocytes comprising administering an effective amount of prieurianin.

Another aspect of the present invention relates to a body weight reducing composition for use in an obese subject comprising a limonoid such as prieurianin. In certain embodiments, the subject comprises a mammal.

In another aspect, the invention relates to a pharmacological composition for probing the biochemistry and physiology of adipogenesis comprising a limonoid.

Yet another aspect of the present invention relates to a method for stimulating phospholipid transfer protein (PLTP) transactivation comprising using prieurianin to induce weight reduction and adiposity in a subject.

Also provided is a method for inhibiting the proliferation of preadipocytes and for preventing the differentiation of preadipocytes into mature adipocytes in a subject, the method comprising administering an effective amount of prieurianin to the subject. In certain embodiments, the subject is considered obese.

Also provided is a method for causing either de-differentiation or for inhibiting accumulation of lipids in differentiated mature adipocytes. The method includes administering an effective amount of prieurianin to the subject.

In yet another aspect, the present invention relates to one or more biomarkers for adipogenesis. In certain embodiments, the biomarker comprises phospholipid transfer protein (PLTP).

Also provided is a method for regulating PLTP gene expression comprising administering an effective amount of prieurianin.

Also provided is a method for blocking transactivation of PLTP by prieurianin administering an effective amount of staurosporine.

Various objects and advantages of this invention will become apparent to those skilled in the art from the following detailed description of the preferred embodiment, when read in light of the accompanying drawings.

### BRIEF DESCRIPTION OF THE DRAWINGS

The patent or application file contains at least one drawing executed in color. Copies of this patent with color drawings will be provided by the United States Patent and Trademark Office upon request and payment of necessary fee. FIG. 1. Pharmacological response of HepG2 cells to topotecan by DNA Microarray. HepG2 cells were treated with 500 nM for various times (A) or with various concentrations of topotecan (B) for 24 hrs. Dendrogram of the expression changes of the phospholipid transfer protein (PLTP) is shown. (C) Induction of PLTP expression by topotecan was confirmed independently by Northern blot analysis.

FIG. 2. Transactivation of PLTP promoter by topotecan—

FIG. 2A. A 1.5 Kb PLTP promoter fused to a luciferase reporter was transactivated by topotecan dose-dependently.

FIG. 2B. Activation of PLTP promoter by topotecan in HepG2 transgenic cells containing the PLTP-promoter luciferase reporter gene fused to the neomycin selectable marker cassette and stably transfected into HepG2 cells to generate the transgenic line (Top panel). Dose-dependent induction of PLTP promoter by topotecan (Bottom panel). Results are the means S.E. of three experiments after normalization with Renilla luciferase.

FIG. 3. Transactivation of PLTP promoter by prieurianin—

FIG. 3A. Dose-dependent transactivation of the PLTP promoter by prieurianin in the HepG2 transgenic cells. Results are the means S.E. of three experiments after normalization with Renilla luciferase.

FIG. 3B. Inhibition of prieurianin transactivation of PLTP promoter by staurosporine. The transgenic HepG2/PLTPpLuc cells were treated with prieurianin either in the presence or the absence of staurosporine. 1, Control; 2. DMSO; 3, 200 nM staurosporine; 4, 5 and 6, 500, 1000 and 2000 nM prieurianin respectively; 7, 8 and 9, 500, 1000 and 2000 nM prieurianin+200 nM staurosporine respectively. Results are the means±S.E. of triplicate experiments.

FIG. 4. Effects of prieurianin on blood insulin, glucose and NEFA levels. Normal and ob/ob mice were treated with 5 mg/kg of prieurianin and serum samples were then collected for (FIG. 4A) insulin; (FIG. 4B) glucose; and (FIG. 4C) non-esterified fatty acid (NEFA) profiling. Results are means of three animals per group for each treatment. Blue column, normal C57BL/6J; and red column, leptin-deficient ob/ob mice.

FIG. 5. Effects of prieurianin on adiposity in ob/ob mice. The leptin-deficient ob/ob mice were treated with 5 mg/kg of prieurianin and subcutaneous and visceral adipose tissues were excised and weighed. Results are means of three to five animals per group as indicated (n).

FIG. 6. Inhibition of NIH-3T3/L1 preadipocytes proliferation by prieurianin. Cells were treated with various concentrations (0.5, 1, and 2 μM) of prieurianin and growth was assessed by counting daily for 7 days. Results are means±S.E. of triplicate experiments.

FIG. 7. Inhibition of preadipocyte differentiation by prieurianin. NIH-3T3/L1 cells were induced into differentiation and simultaneously treated with 2 μM of prieurianin. Differentiated cells were stained with oil red O—FIG. 7A, undifferentiated control.

FIG. 7B. Differentiated adipocytes.

FIG. 7C. Induction of differentiation in the presence of 2 μM prieurianin.

FIG. 7D. Flow cytometric analysis of annexin V binding to phosphatidylserine, apoptotic cells are on the upper right quadrant. All experiments were conducted in triplicate.

FIG. 8. Prieurianin induced loss of differentiated adipocytes. NIH-3T3/L1 preadipocytes were induced into differentiation. Five days following differentiation, cells were treated with various concentrations (0.5, 1, and 2 μM) of prieurianin for an additional five days and followed by staining with oil red O. Micrographs were obtained from representatives of triplicate experiments. Isopropanol extracts of positively stained cells were quantified spectrophotometrically at 510 nm as shown in histogram on the right. Results are means±S.E. of triplicate experiments.

FIG. 9. Staurosporine blocks prieurianin induced differentiation of preadipocytes. Preadipocytes differentiated in prieurianin (0.5, 1 or 2 μM) were together treated either with or without 200 nM staurosporine. Micrographs showed oil red O stained cells 12 days post-induction.

FIG. 9A, undifferentiated;

FIG. 9B, differentiated;

FIG. 9C, differentiated in 2 μM prieurianin;

FIG. 9D, differentiated in 2 μM prieurianin together with 200 nM staurosporine.

Histogram represents A510 nm absorbance of oil red O stain isopropanol extracts from cells. Results are means±S.E. of triplicate experiments.

FIG. 10. Release of adiponectin and PLTP by preadipocytes and adipocytes, and in serum of normal mice. Preadipocytes (B and D) were treated with 2 μM of prieurianin and media collected after 36 hrs for Western blot analysis of either adiponectin (A and B) or PLTP (C and D). Alternatively, five days following differentiation, conditioned media of adipocyets (A and C) were collected for Western analysis. E. Serum of normal C57BL/6J mice given prieurianin or topotecan (0, 2, 5, and 10 mg/kg) was analyzed by Western blot for PLTP.

FIG. 11. Transactivation of PLTP promoter by cytotoxic and non-cytotoxic drugs. Survey of transactivation of the PLTP promoter by various cytotoxic and non-cytotoxic drugs in the HepG2 transgenic cells was conducted. Results are the means S.E. of three experiments.

FIG. 12. Effects of trichostatin A (TSA) on transactivation of PLTP promoter by prieurianin. Dose response activation of PLTP promoter by prieurianin either in the presence or the absence of 200 nM TSA. Results are the means S.E. of three experiments.

FIG. 13. Effects of prieurianin treatment on body weight and food intake in normal C45BL/6J or the C57BL/6J leptin-deficient ob/ob mice.

FIG. 14. Effects of prieurianin treatment on serum lipoproteins, PLTP activity, and leptin levels in normal C57BL/6J or the C57BL/6J leptin-deficient ob/ob mice.

FIG. 15. Effects of prieurianin in db/db and diet-induced obese Ceacam^{-/-} mice-.

FIG. 15A. Groups of 10 db/db mice were given either 3 or 5 mg/kg of prieurianin i.p. daily for 30 days.

FIG. 15B. Genetically diabetic Ceacam^{-/-} knockout mice were fed high fat diet for fattening for 4 weeks followed by daily prieurianin treatment (3 or 5 mg/kg) for 21 days. Vehicle treated db/db and Ceacam^{-/-} mice were given equivolume of Captisol. Results are the means S.E. of 10 animals per group.

FIG. 16. Effects of prieurianin in diet-induced obese C57Bl/6J mice. B6 mice were put on 60% kcal high fat diet for approximately 15 weeks and then divided into groups of 10 and were then treated with either 1 (green) or 3 (brown) mg/kg of prieurianin daily intraperitoneally for three weeks compared to untreated (blue) or vehicle-treated (red) controls.

FIG. 16A. Average weight changes of mice treated with prieurianin compared to controls through three weeks of treatment.

FIG. 16B. Average food consumption in B6 mice treated with prieurianin as in FIG. 16A.

FIG. 16C. Average weight changes in B6 mice treated with prieurianin on an “on-off” cyclical schedule for a total of 4 cycles described in the text. Results are the means S.E. of 10 animals per group, and the 3 mg/kg group with 20 mice.

FIG. 17. An “on-off” or “cyclical” treatment schedule for overcoming drug-induced tolerance. This treatment strategy comprise of specified doses of treatment for a specified duration of treatment coupled with intermittent drug holiday, can overcome drug induced tolerance, desensitization, or lack of response in the treatment of metabolic disorders and other disorders.

FIG. 18. Effects of prieurianin on C/EBPα and β and PPARγ mediated transcription in adipogenesis. Response elements corresponding to C/EBPα and β and PPARγ are cloned into the pGL3 basic luciferase reporter plasmid. L1 preadipocytes were contransfected with the reporter plasmid either in the presence or the absence of the corresponding transcription factors cloned into expression vector, followed by treatment with 2 μM of prieurianin. Cells were harvested for luciferase assay 15-24 hrs later. Results are means±S.E. of triplicate experiments.

FIG. 19. Effects of bufalin on preadipocyte differentiation, and dedifferentiation/delipidation in adipo-cytes. NIH-3T3/L1 cells were induced into differentiation and simultaneously treated with either 1 μM of bufalin or 2 μM of prieurianin. Differentiated cells were stained with Nile Red. Alternatively, preadipocytes were induced into differentiation and five days following differentiation, cells were treated with either 1 μM of bufalin or 2 μM of prieurianin for an additional five days and followed by staining with Nile Red. Stained cells were evaluated by fluorescence microscopy.

### DETAILED DESCRIPTION OF THE PREFERRED EMBODIMENT

The present system provides a method for the induction of the PLTP gene expression. The present system also provides a method for raising PLTP levels and modulating reverse cholesterol transport.

In another aspect, there is provided non-cytotoxic natural product small molecules that raise PLTP levels and modulate reverse cholesterol transport. siRNA-induced loss of PLTP in C. elegans increases fat storage. It is now discovered that prieurianin transactivates PLTP gene expression and is a feeding deterrent.

In another aspect, there is provided herein a method of using prieurianin as a novel pharmacological composition useful for probing the biochemistry and physiology of adipogenesis.

Prieurianin induces PLTP gene expression and effectively reduces body weight and fat mass. Prieurianin also inhibits the proliferation and differentiation of preadipocytes, and either causes adipocytes to de-differentiate or prevents the adipocytes from accumulating lipids. Due to the effects of prieurianin on the adiposity of ob/ob mice and its anti-adipogenic effects on cultured preadipocytes and adipocytes, the inventor now believes that PLTP is required for the anti-adipogenic effects of prieurianin on body weight and fat mass reduction.

In another aspect, there is provided a method of using prieurianin as an effective anti-obesity drug. Its efficacy in mice was tested and prieurianin significantly reduced total body weight, fat and food intake. The drug also reversed the hyperglycemic state of the mice to levels comparable to normal mice.

In further molecular studies, it was determined that prieurianin inhibits the proliferation of preadipocytes, and also prevents their differentiation into adipocytes. Prieurianin is capable of either causing de-differentiation of the adipocytes or preventing them from accumulating lipids.

Prieurianin inhibits the release of adiponectin by preadipocytes, thus may account for the block of their differentiation into adipocytes. Paradoxically, while prieurianin induces the secretion of PLTP in adipocytes, the release of PLTP is not inhibited in preadipocytes. In cell culture studies, prieurianin is relatively non-cytotoxic compared to topotecan (data not shown), and no overt toxicity was observed in animals given the drug for the duration of the experiments.

Thus, prieurianin is a natural product small molecule with anti-obesity effects that target adipogenesis.

In a particular aspect, prieurianin is shown herein to have an effect on producing weight loss in mouse models of obesity with various underlying pathogenic mechanisms by suppressing appetite, and additionally through its unique pharmacological profile in inhibiting the proliferation and differentiation of preadipocytes, causing dedifferentiation and delipidation of adipocytes.

In addition, it is now shown herein that the molecular mechanisms of prieurianin is now believed to reside in its ability to inhibition the transcription regulation of adipogenesis by activating the NFκB signaling pathway and by inhibiting C/EBPα and β, or PPARγ mediated transcriptional activation of preadipocytes differentiation into adipocytes.

The above described advantages will now be illustrated by the following non-limiting examples.

### Example 1

### Pharmacological Response of HepG2 Cells to Topotecan

The mechanisms of resistance to topoisomerase (Top) 1 inhibitors by expression genomics were studied by conducting time-course and dose-response experiments by DNA microarray to investigate the pharmacological response of the human hepatocellular blastoma HepG2 cells to topotecan, which were either treated with 500 nM of topotecan (a cytotoxic anticancer agent) for various times (0, 1, 3, 5, 10, 15, and 24 hrs), or with various doses of topotecan (0, 10, 50, 100, 300, 500, and 1000 nM) for 24 hrs.

Overall gene expression changes induced by topotecan were modest, with most genes exhibiting low level alterations in expression, except for the PLTP gene.

Results in FIG. 1 showed the dendrogram of the time course (FIG. 1A) and dose response (FIG. 1B) expression of PLTP in response to topotecan. Activation of PLTP expression by topotecan was temporally regulated and dose dependent, with a late onset, peaking at 24 hrs with an approximately 20-fold induction. Topotecan-induced PLTP expression was validated independently by Northern blot analysis, which showed that PLTP was induced by topotecan dose-dependently (FIG. 1C), consistent with those observed in the microarray studies.

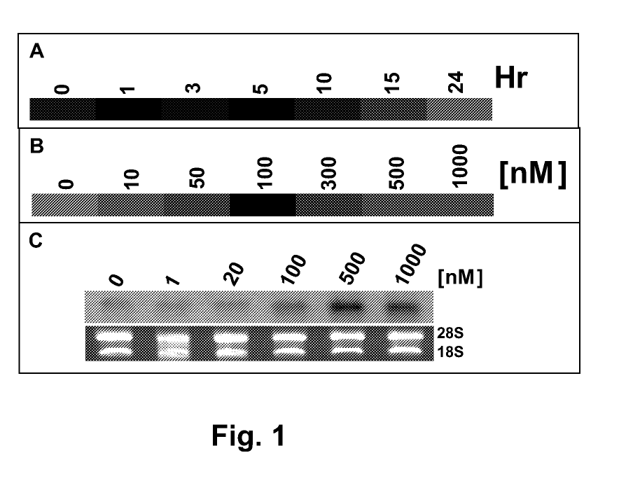

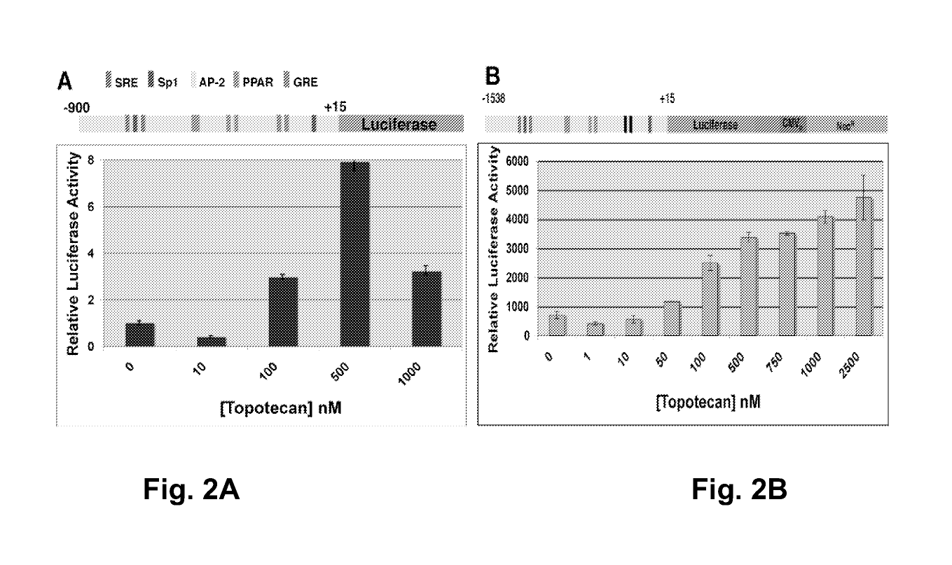

The induction of PLTP gene expression is transcriptionally regulated by topotecan as the promoter of PLTP fused to a luciferase reporter is transactivated by topotecan dose dependently (FIG. 2A), blot analysis.

Thus, Top1 inhibitors induce PLTP gene expression in HepG2 cells in culture (see FIGS. 1 and 2) as well as in vivo in mice (data not shown). In addition, the inventor herein now shows that PLTP is useful as a biomarker for obesity and has an important role in adipogenesis in obesity.

### Example 2

### Screening for Natural Product Small Molecule Inducers of PLTP Gene Expression

PLTP is involved in reverse cholesterol transport. Also, PLTP expression and activity is associated with obesity. In addition, an increase in fat storage in C. elegans following inactivation of PLTP gene expression by RNA-mediated interference shows that small molecules that target PLTP are may be useful to develop drugs for treating obesity.

To determine whether non-cytotoxic small molecules could induce PLTP expression, the inventor herein subcloned the PLTP-promoter luciferase reporter into a vector containing a neomycin (G418)-resistance selectable marker and generated a transgenic HepG2 cell line, which harbors the PLTP-promoter luciferase reporter, by stable gene transfection and selection with G418. The transgenic cell line, HepG2/PLTPpLuc exhibits topotecan response that was similar to HepG2 cells transiently transfected with the PLTP-promoter reporter (FIG. 2B).

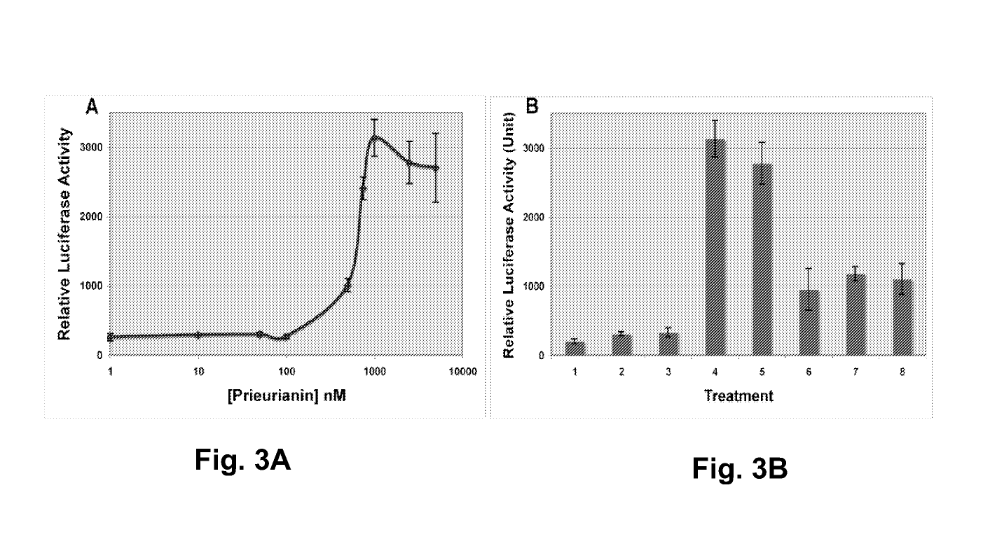

The transgenic cells were then screened with a library of small molecules derived from natural products. Prieurianin exhibited the strongest transactivation of the PLTP promoter, and showed induction of PLTP in a dose-dependent manner (FIG. 3A).

The inventor herein found further that the transactivation of PLTP promoter activity by prieurianin was inhibited by staurosporine, thus suggesting that the transcriptional regulation of PLTP expression by prieurianin is regulated by a protein kinase (FIG. 3B).

### Example 3

### Anti-Obesity Effects of Prieurianin

Little is known about prieurianin. It is a limonoid compound and a natural product anti-feedant that exhibit antagonism against 20-hydroxyecdysone activity in drosophila cells in culture. The drug is relatively non-cytotoxic compared with topotecan in cell culture studies.

Prieurianin was administered intraperitoneally to 12-14 week-old normal C57BL/6J mice and the genetically leptin-deficient ob/ob mice (2 or 5 mg/kg) twice a week for two weeks. Controls received equivolume injections of drug vehicle. Body weight and food intake were measured every three days, and blood samples were collected at the end of the experiment.

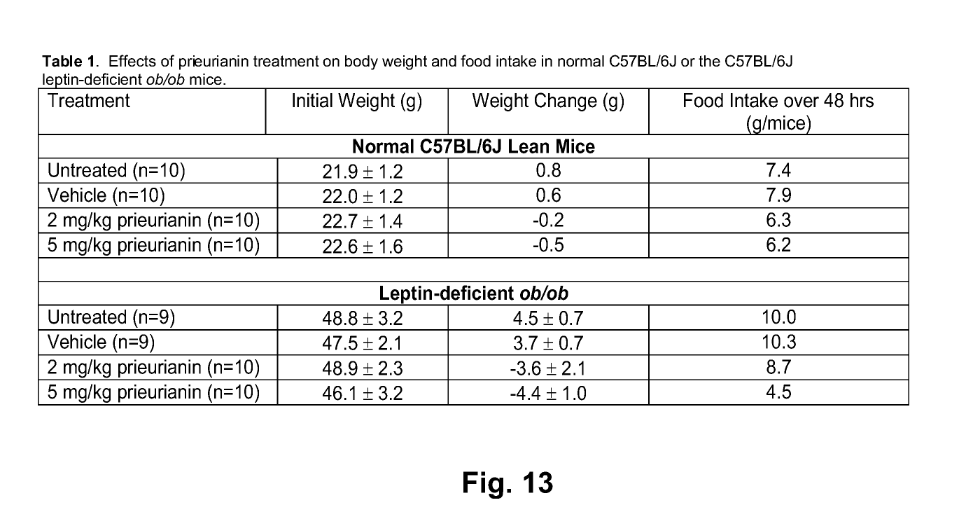

Treatment with prieurianin resulted in a dose dependent reduction of up to 10% in total body weight for either 2 or 5 mg/kg treated leptin-deficient ob/ob mice after two weeks (see FIG. 13 containing Table 1).

In addition, a dose dependent decrease in food intake, by as much as 50%, was also observed in the 5 mg/kg treated group relative to the untreated or vehicle treated controls. A modest body weight loss as well as reduced food intake was also observed in the normal C57BL/6J mice. These results suggest that the resulting weight loss and decrease in food intake is attributed to the feeding deterrent effects of prieurianin.

### Example 4

### Metabolic Effects of Prieurianin

Obesity contributes to hypertension, high serum cholesterol, low HDL cholesterol, and hyperglycemia, thus potentially leading to higher risk of cardiovascular disease. Abdominal obesity especially correlates with metabolic risk factors. The leptin-deficient ob/ob mice are hyperlipidemic, and hyperglycemic. To test whether prieurianin altered the metabolic or endocrinological parameters in addition to appetite, the serum lipid profile, insulin and glucose levels were measured. However, no significant changes in the triglyceride levels were observed in both prieurianin treated and untreated normal controls as well as the ob/ob mice (data not shown).

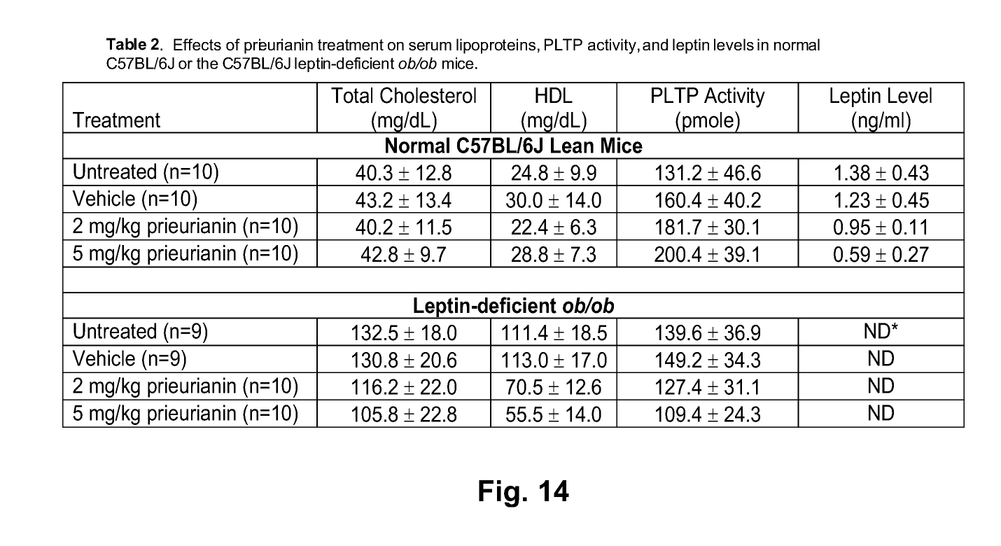

Total cholesterol and HDL levels also remained relatively unchanged with or without treatment with prieurianin in the normal untreated and vehicle treated mice (see FIG. 14 containing Table 2).

In contrast, though modest, total cholesterol level was significantly lower in prieurianin treated ob/ob mice. Furthermore, HDL levels were approximately two-fold lower in prieurianin treated ob/ob mice than untreated and vehicle-treated animals. Treatment with prieurianin also caused an increase in serum PLTP activity in the normal C57BL/6J mice (FIG. 14).

Paradoxically, ob/ob mice given prieurianin showed a decrease in serum PLTP activity, even though prieurianin activates the expression of PLTP gene (FIG. 3). However, the decrease in PLTP activity in prieurianin treated ob/ob mice is consistent with those reported in human obese subjects following weight loss. In normal mice, treatment with prieurianin caused a decrease in leptin levels, but which was not detectable in ob/ob mice as expected (FIG. 14).

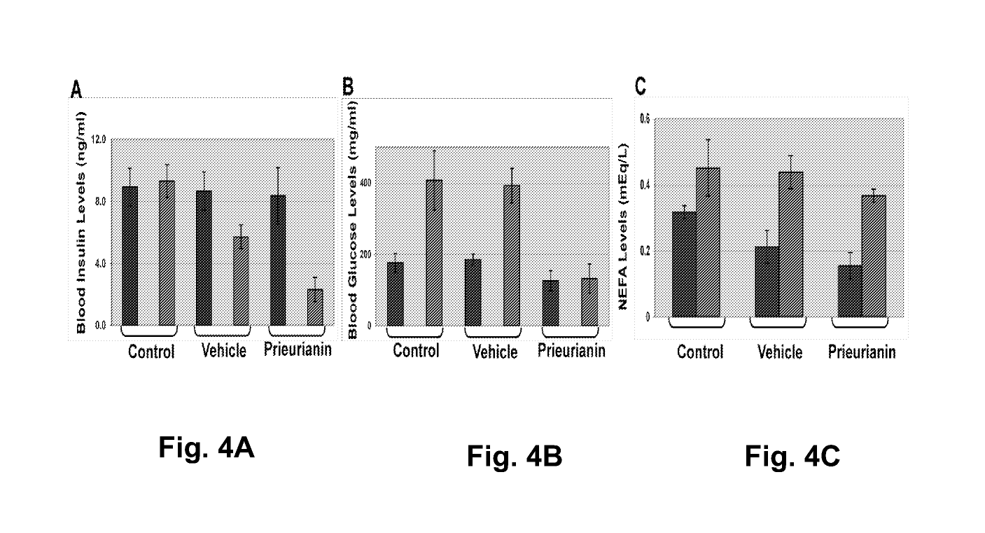

Hyperglycemia in the ob/ob mice was reversed to levels comparable to those of normal controls in prieurianin (5 mg/kg) treated ob/ob mice (see FIG. 4B).

In ob/ob mice, prieurianin also caused insulin levels to reduce by approximately three to four-fold (see FIG. 4A). Moreover, prieurianin treatment did not alter the insulin or the glucose levels significantly in normal C57BL/6J mice.

Disturbances in pathways of lipolysis and fatty acid regulation are of importance in the etiology of obesity. Alteration in the uptake of non-esterified fatty acid (NEFA) by skeletal muscle and adipose tissue is a critical determinant of its concentration in the plasma. As shown in FIG. 4C, the NEFA level is higher in the leptin-deficient ob/ob mice compared to normal C57BL/6J mice. Treatment with prieurianin in normal mice resulted in a decrease in NEFA levels. However, in the ob/ob mice, only a modest decrease in NEFA levels was observed after the administration of prieurianin.

### Example 5

### Effects of Prieurianin on Adipogenesis

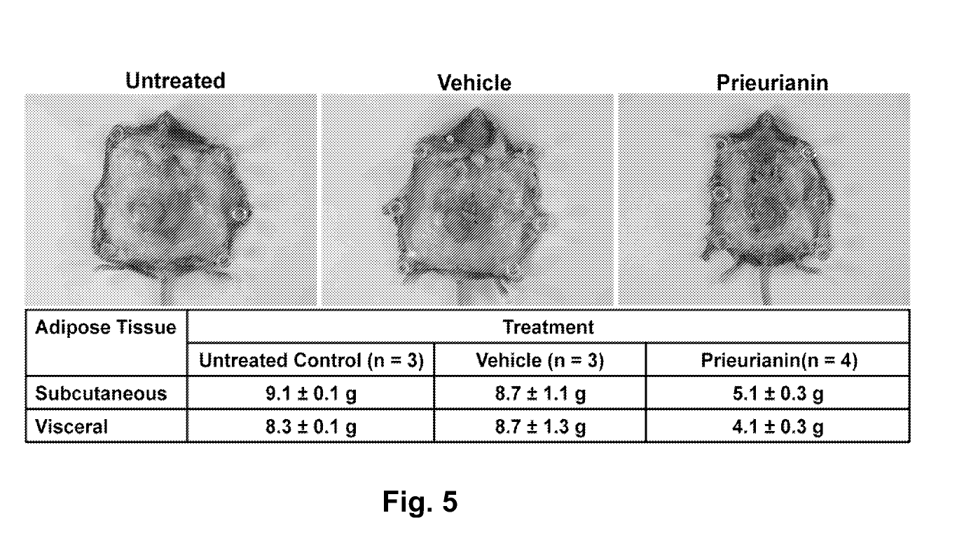

To further examine the effects of prieurianin on the adiposity of the normal C57BL/6J and the ob/ob mice, we measured their visceral and subcutaneous body fat. The total body fat was significantly reduced by greater than 50% in the prieurianin treated ob/ob mice compared to the untreated and vehicle-treated controls (see FIG. 5). The percent body fat of the normal C57BL/6J mice was not significantly altered by prieurianin at any dose (data not shown).

It has been shown previously that the differentiation of preadipocytes into mature adipocytes in culture is completely inhibited by TNFα, IL-1β, IFNγ, or TGFβ1, which is accompanied by abolition of the release of adiponectin. Since prieurianin significantly reduced both the subcutaneous and visceral adipose tissues in ob/ob mice (see FIG. 5), the inventor herein examined in cell culture studies its effects on the (i) proliferation of preadipocytes; (ii) differentiation of preadipocytes into adipocytes; and (iii) inhibition of lipid accumulation or promotion of dedifferentiation of adipocytes.

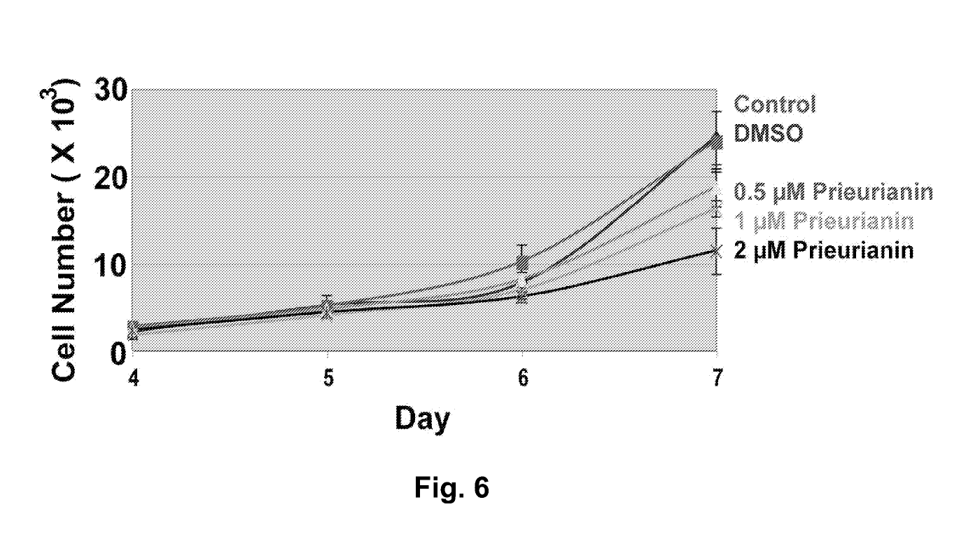

As shown in FIG. 6, prieurianin inhibited the proliferation of the NIH-3T3/L1 preadipocytes in a dose-dependent manner. A pronounced inhibition (50%) was observed at 2 μM of prieurianin on day 7.

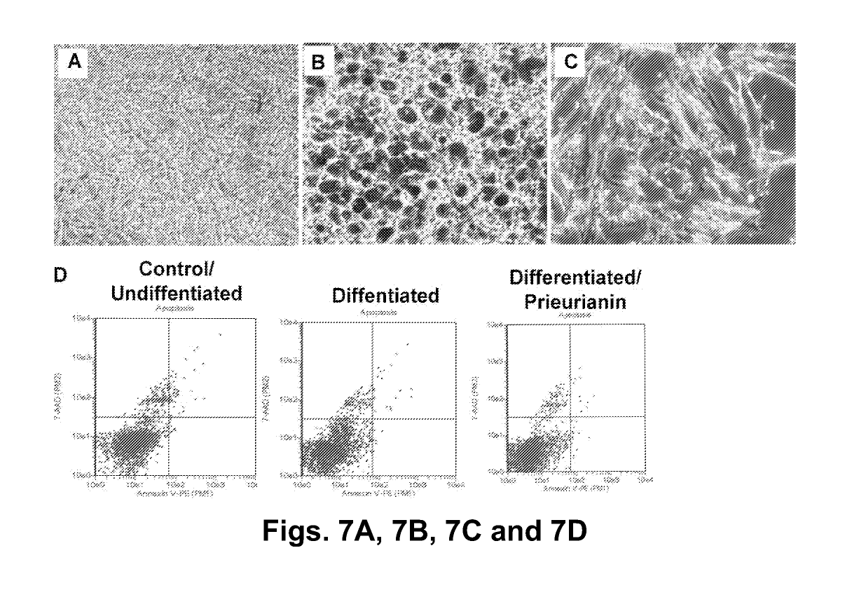

To determine the effects of prieurianin on the differentiation of preadipocytes to adipocytes, the NIH-3T3/L1 preadipocytes were treated with or without the drug at the same time when induction of differentiation was initiated. We found that prieurianin also dose-dependently prevented the differentiation of preadipocytes into the lipid accumulating adipocytes, as evident from the marked reduction in the number of oil red O stained lipid accumulating adipocytes relative to the untreated/undifferentiated and differentiated controls (see FIGS. 7A-C).

Interestingly, prieurianin treated preadipocytes acquired a rather different morphology compared to the preadipocytes (FIG. 7C), and did not differentially induce apoptosis in the preadipocytes as indicated by the lack of annexin V binding to phosphatidylserine (see FIG. 7D). Cell numbers were also relatively comparable (data not shown) between the untreated or vehicle treated controls and the drug treated differentiating cells.

Results indicated that prieurianin inhibits the proliferation of preadipocytes, and also prevents the differentiation of preadipocytes into mature adipocytes. To assess whether prieurianin has any effect on the differentiated adipocytes, the preadipocytes were allowed to differentiate into adipocytes and then were further cultured for about five days before treating the adipocytes with prieurianin for an additional five to six days, followed by oil red O staining for the presence of lipid accumulating adipocytes.

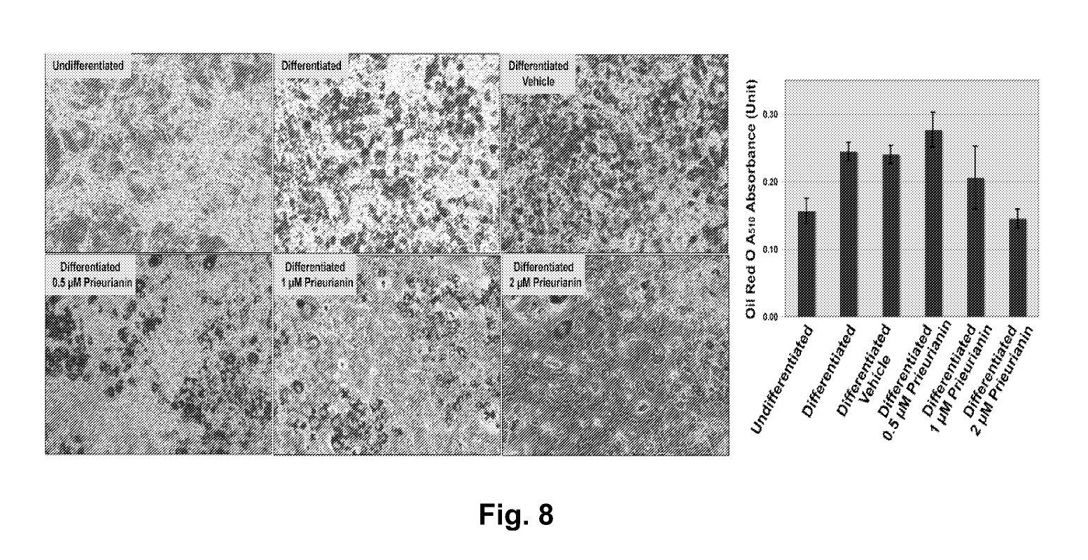

A profound dose-dependent reduction in the number of oil red O stained cells in prieurianin treated cells (see FIG. 8, bottom panels) was observed, whereas vehicle treated cells showed comparable number of lipid positive staining cells as the differentiated control. When quantified by oil red O stain A510 absorbance, we showed that at 2 μM of prieurianin, the number of lipid positive cells was almost absent and reduced to a level comparable to the undifferentiated control (see FIG. 8, histogram on right). These results suggest further that, in addition to inhibiting preadipocytes proliferation and preventing their differentiation, prieurianin also acts on differentiated mature adipocytes by causing either their de-differentiation or inhibiting their accumulation of lipid.

### Example 6

### Staurosporine Partially Reversed the Inhibition of Differentiation by Prieurianin

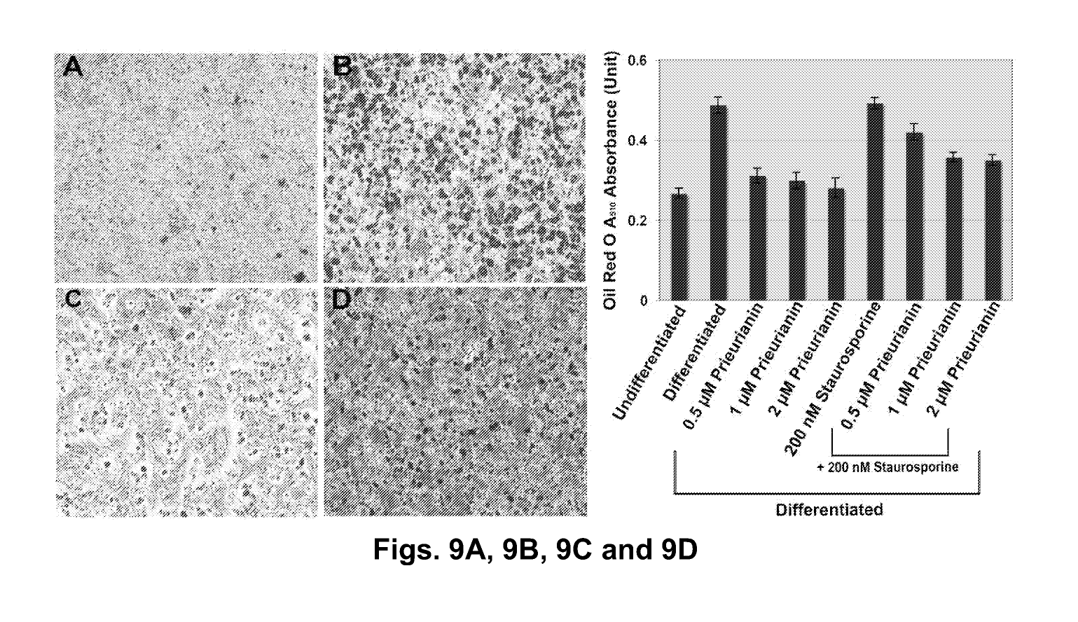

Transactivation of PLTP by prieurianin can be blocked by staurosporine (see FIG. 3B), a potent inhibitor of the “conventional” protein kinase C (PKC) isozymes. We also showed that transcriptional activation of PLTP by prieurianin inhibits preadipocytes differentiation (see FIG. 7). To assess whether inhibiting prieurianin mediated transactivation of PLTP by staurosporine might reverse the block of differentiation, differentiation of preadipocytes was induced in the presence of prieurianin (0, 0.5, 1 and 2 μM) either together with or without 200 nM staurosporine. Consistent with the above results (see FIG. 7), in the absence of staurosporine, 2 μM of prieurianin almost completely inhibited preadipocytes differentiation (see FIG. 9C).

The addition of staurosporine at the time of differentiation induction partially reversed the inhibition by prieurianin (see FIG. 9D), whereas staurosporine alone showed no observable effects on differentiation (data not shown). These results were confirmed by monitoring the A₅₁₀ absorbance of the isopropanol extract of oil red O stained preadipocytes. Prieurianin inhibited preadipocytes differentiation in a dose dependent manner, and the inhibition was partially reversed by staurosporine (see FIG. 9, histogram). Thus, it is now believed by the inventor herein thus suggest that PLTP expression induced by prieurianin is required for the inhibition of preadipocytes differentiation.

### Example 7

### Effects of Prieurianin on the Secretion of Adipokines and PLTP

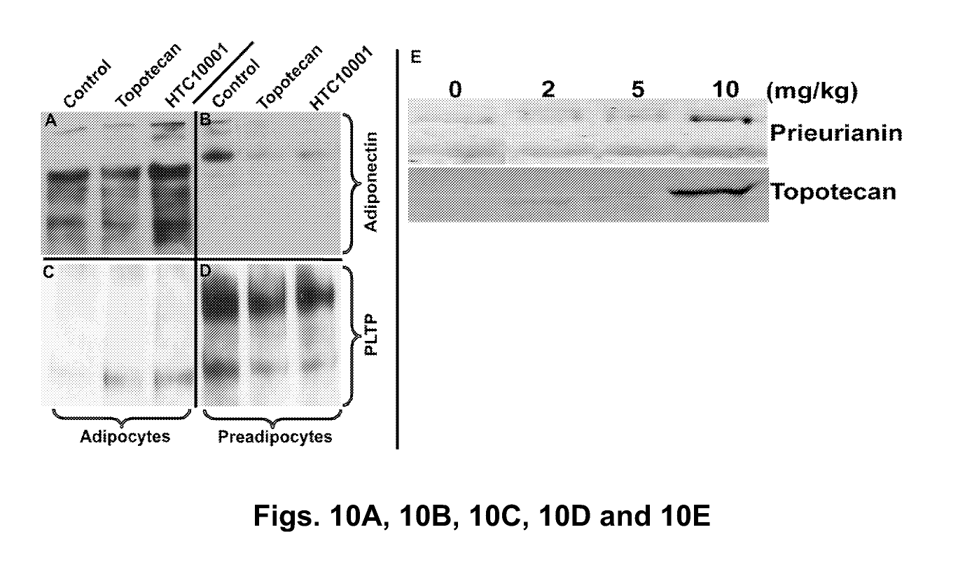

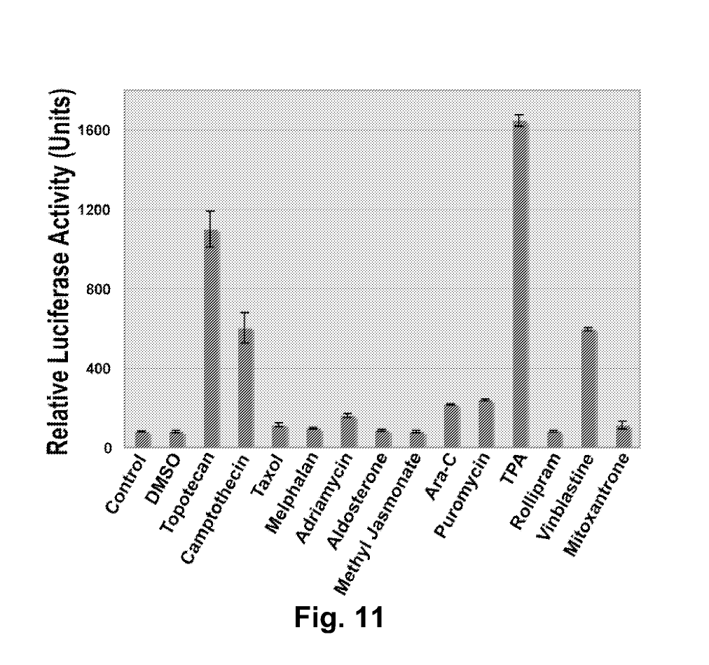

Adipose tissue contains various types of cells including preadipocytes and adipocytes. Also, preadipocytes secrete factors involved in their own differentiation. Once differentiated, the mature adipocytes acquire the ability to communicate distally with other organs including brain, liver, and skeletal muscle and locally with other cells such as preadipocytes, endothelial cells and monocytes/macrophages by secreting leptin and adiponectin. In addition, anti-adipogenic cytokines prevent the release of adiponectin by preadipocytes. Thus, the production of adiponectin and PLTP by preadipocytes and adipocytes was assessed. An inhibition of adiponectin release into the conditioned culture media by preadipocytes, approximately 36 hrs following treatment with prieurianin (see FIG. 10B), was observed.

Topotecan, which induces the expression of PLTP (see FIG. 10), also significantly inhibited the production of adiponectin by the NIH-3T3/L1 preadipocytes (FIG. 10B). These results are consistent with previous reports that blockage of preadipocyte differentiation is accompanied by an inhibition in the release of adiponectin. In addition, prieurianin, but not topotecan, induced the production and release of high molecular weight form, as well as a modest increase in the secretion of total adiponectin in differentiated adipocytes (see FIG. 10A).

The expression and secretion of PLTP by preadipocytes and adipocytes was also assessed. Thus, preadipocytes produced and released both the low and the high molecular weight forms of PLTP, while adipocytes secreted only the low molecular weight form (see FIGS. 10C and 10D) into the conditioned media.

Unexpectedly, following treatment with either topotecan or prieurianin in preadipocytes, release of the low but not the high molecular form of PLTP was reduced (see FIG. 10D).

In contrast, topotecan and prieurianin induced an increase in the release of only the low molecular weight form of PLTP in adipocytes (see FIG. 10C). These differential profiles in the release of PLTP between preadipocytes and adipocytes suggest a complex endogenous physiological response to the pharmacological effects of prieurianin.

### Example 8

### Serum PLTP Protein Levels Following Prieurianin and Topotecan Treatment

Time-course and dose-response studies with topotecan in the microarray analysis showed that PLTP is a late gene with an onset of induction approximately 12-15 hrs following treatment with the drug (see FIG. 10A). The onset of PLTP transactivation by prieurianin is similar to that of topotecan. The induction of PLTP gene expression by either prieurianin or topotecan (0, 2, 5, and 10 mg/kg) was accompanied by a rise in the serum PLTP protein levels (see FIG. 10E).

### Example 9

### Pharmacological Inhibition of PLTP Transactivation by Prieurianin on Weight Reduction and Adiposity in Mice

The pharmacological inhibition of prieurianin with staurosporine on adiposity in normal and ob/ob mice was examined. The PKC activator, 12-O-tetradecanoylphorbol-13-acetate (TPA), induced PLTP promoter (see FIG. 11), and the activation was abolished by the PKC inhibitor staurosporine. The inventor herein now believes that the PKC signaling pathway may be involved in the transcriptional regulation of PLTP expression. These results also showed that prieurianin induced PLTP promoter activity was inhibited by staurosporine (see FIG. 3B).

In addition, inhibition of preadipocytes differentiation by prieurianin was partially reversed by staurosporine (see FIG. 9), further pointing to a potential role for PKC in the transactivation of PLTP and inhibition of differentiation by prieurianin. Since transactivation of PLTP by prieurianin was blocked by staurosporine, these results show that prieurianin induced weight loss and inhibition of adipogenesis can be abrogated or reversed by co-administration of staurosporine.

### Example 10

### Pharmacological Inhibition of Prieurianin Induced PLTP Expression by Staurosporine on Adipogenesis

The adipogenic transcription factors peroxisome proliferator-activated receptor-γ (PPARγ) and CCAAT/enhancer binding protein-α and β (C/EBPα and β) play key role in the complex transcriptional cascade that occurs during adipogenesis. The interaction between PPARγ and RB decreases the transcriptional activity of PPARγ through recruitment of the histone deacetylase, HDAC3. Inhibition of HDAC activity consequently results in a strong activation of PPARγ. Valproic acid has been shown to inhibit adiponectin gene expression in mice and in the NIH-3T3/L1 preadipocytes and decreases C/EBPα protein levels and its binding to the adiponectin promoter. Since prieurianin is a transcriptional activator of PLTP, the inventor herein now believes that some of the pharmacological effects of the drug are influenced by these transcription factors.

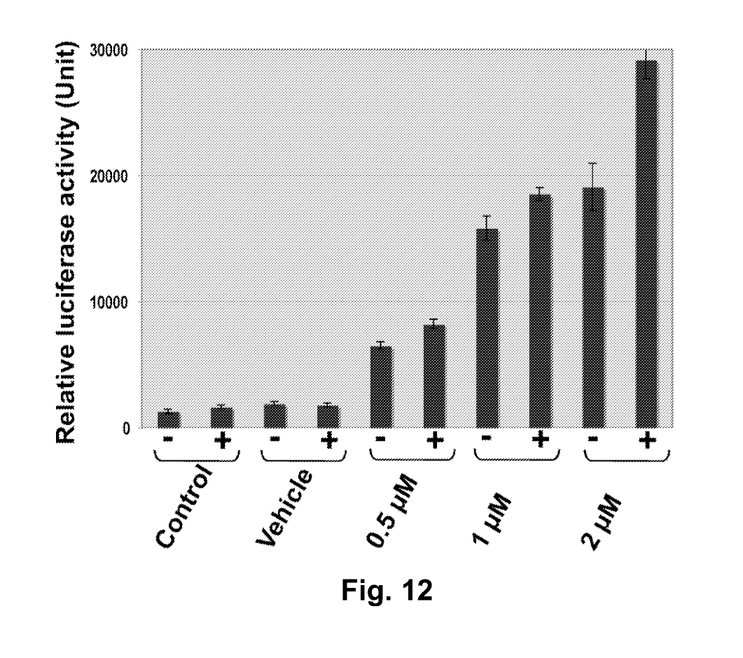

As shown in FIG. 12, the HDAC inhibitor trichostatin A (TSA), though modest, potentiated the transactivation of PLTP promoter activity by prieurianin, thus showing that PPARs may play a role in the transcriptional regulation of prieurianin. These preliminary results also showed that staurosporine, a PKC inhibitor, strongly inhibited PLTP transactivation by prieurianin (see FIG. 3B) and also reversed the inhibition of proliferation and differentiation of preadipocytes by prieurianin (see FIGS. 6 and 7). While there are some similarity on the inhibition of preadipocytes differentiation by prieurianin and endothelin-1, the pharmacological effects of staurosporine on prieurianin and endothelin-1 are not the same, thus showing that endothelin-1 and prieurianin inhibited differentiation of preadipocytes, probably through different pathways.

### Example 11

### Anti-Obesity Effects of Prieurianin

The effects of prieurianin were tested in three additional mouse models of obesity including the genetically hyperinsulinemic leptin-receptor deficient db/db, the Ceacam−/− glucose intolerant/diabetic, and the diet-induced obese mice. Prieurianin was administered intraperitoneally (i.p.) to 12-14 week-old db/db mice daily (3 or 5 mg/kg) for 30 days. The diet-induced obese Ceacam−/− diabetic mice were fed a high fat diet for 4 weeks for fattening, followed by prieurianin treatment (3 or 5 mg/kg) for 3 weeks. Vehicle treated controls received equivolume injections of Captisol (CyDex Inc., Lenexa, Kans.). Body weight and food intake were measured every three days, and blood samples were collected at the end of the experiment.

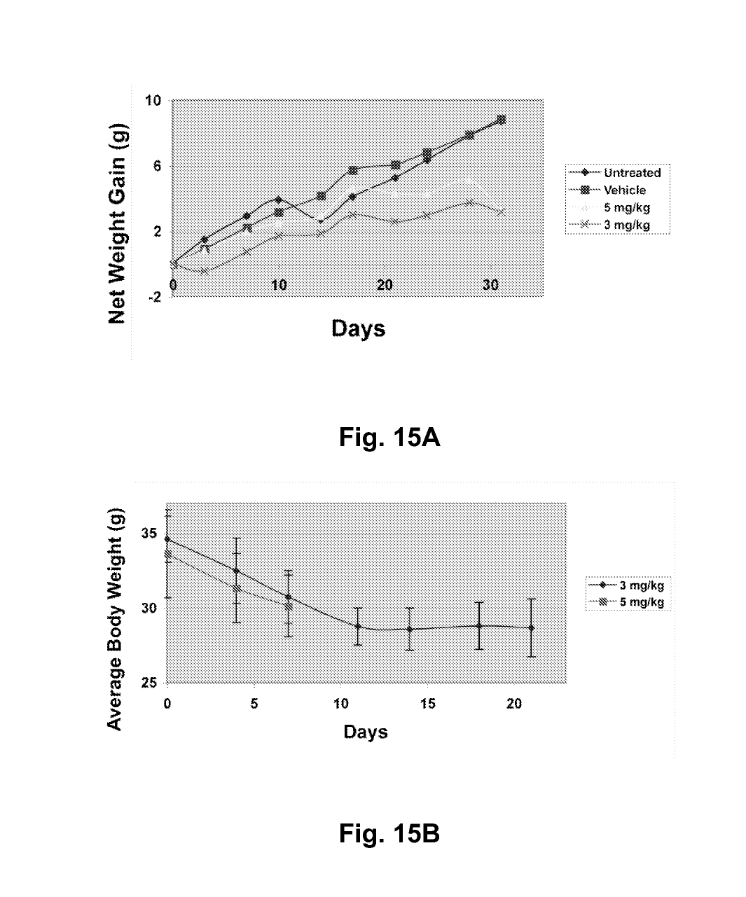

No weight loss was observed in prieurianin treated db/db mice, but instead we found a pronounce attenuation of weight gain by 50% over three weeks compared to untreated or vehicle-treated controls (see FIG. 15A).

In the diet-induced obese Ceacam−/− diabetic mice, approximately 20-26% body weight loss was observed in prieurianin treated animals (see FIG. 15B), accompanied by >50% reduced food intake (data not shown) compared to controls. Body weight returned to pre-fattening level in the 3 mg/kg treated group. Mice given 5 mg/kg prieurianin were euthanized after 14 days of treatment due to severe weight loss and unknown toxicity. Little subcutaneous or visceral fats were observed in postmortem analysis in these mice relative to controls (data not shown).

The dense high-caloric diets coupled with sedentary lifestyles prevalent worldwide in have contributed to the sharp rise in the incidence of obesity. To further test the efficacy of prieurianin in obesity, its effects in the diet-induced obese (DIO) C57BL/6J (B6) mouse model were also investigated.

The B6 mice were fed a 60% kcal high fat diet for approximately 15 weeks to gain weight and then treated with either 1 or 3 mg/kg of prieurianin intraperitoneally daily for 3 weeks. Mice continued to have access to the 60% kcal diet ad libitum during treatment.

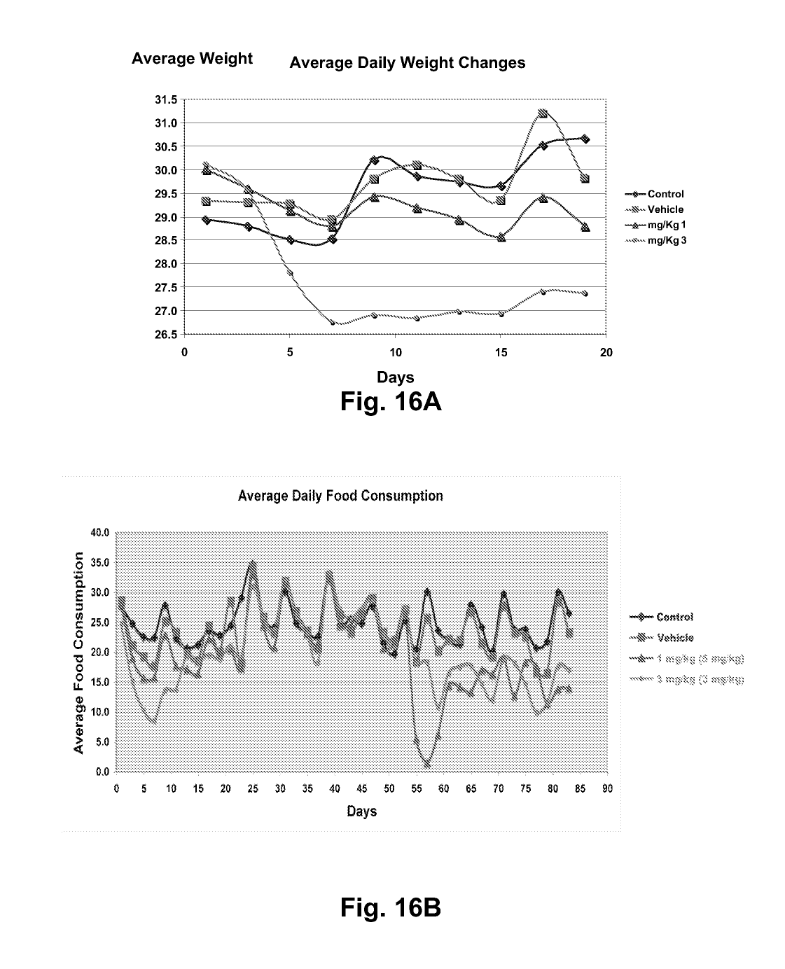

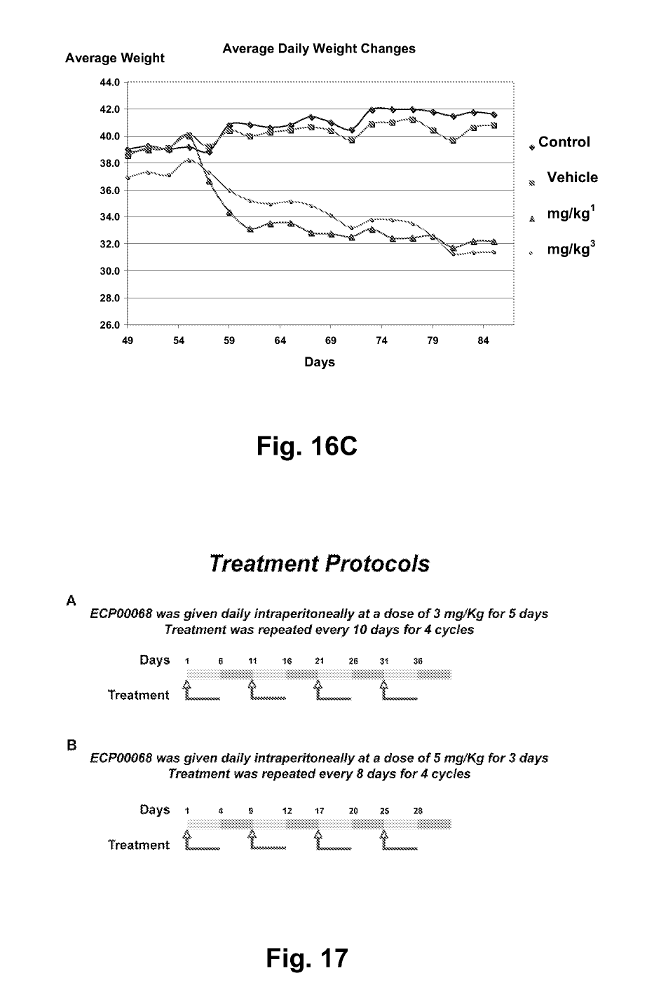

The results showed a dose dependent induction of weight loss of up to 10% of total body weight within seven days of treatment (see FIG. 16A), which was accompanied by 70-80% decrease in food consumption that eventually returned to almost normal levels by the end of the three-week treatment (see FIG. 16B). However, the attained weight loss was followed by a gradual increase in body weight after week two. These results suggested that prieurianin-treated mice might have developed tolerance to the drug.

To circumvent the problem of drug induced tolerance, a new protocol was developed where prieurianin treatment was stopped with the DIO mice, and the mice continued to be maintained on the high fat 60% kcal diet. Four weeks later, the mice were treated again with the following protocol: either 3 mg/kg of prieurianin for 5 days and followed by 5 days of drug holiday (no treatment), with the treatment repeated for 3 more cycles; or 5 mg/kg of prieurianin for 3 days and followed by 5 days of drug holiday, with the treatment repeated for 3 more cycles.

It was observed that this “on-off” treatment strategy (see FIG. 17) seemed to overcome the drug-induced tolerance and resulted in more pronounced response of up to 20% decrease in body weight with either 3 or 5 mg/kg of prieurianin and no observable increase in body weight towards the end of the study (FIG. 16C). Food consumption was reduced by approximately 40% and maintained throughout the duration of the cyclical treatment protocol (FIG. 16B).

The results revealed that the loss of effectiveness of the drug by daily drug treatment can be overcome with this novel cyclical or on-off treatment protocol, which produces a greater response and the maintenance of weight loss, thus circumventing drug-induced tolerance. The inventor herein now believes that the appetite suppressant anti-obesity drugs (including Meridia) and other anti-obesity drugs probably lose their effectiveness over a protracted course of treatment and encounter tolerance due to compensatory physiological hormonal changes in respond to drug treatment that disrupts energy metabolism.

This novel cyclical or on-off treatment protocol is a way to improve the efficacy of anti-obesity drugs and might be applicable in the treatment of metabolic disorders in general and other human disorders.

### Example 12

### Mechanisms of Action of Prieurianin

Prieurianin inhibits the proliferation and differentiation of preadipocytes, and also causes either the de-differentiation or delipidation of adipocytes. To ascertain the molecular mechanisms of prieurianin, the effects of prieurianin on the transcriptional regulation of adipogenesis were evaluated.

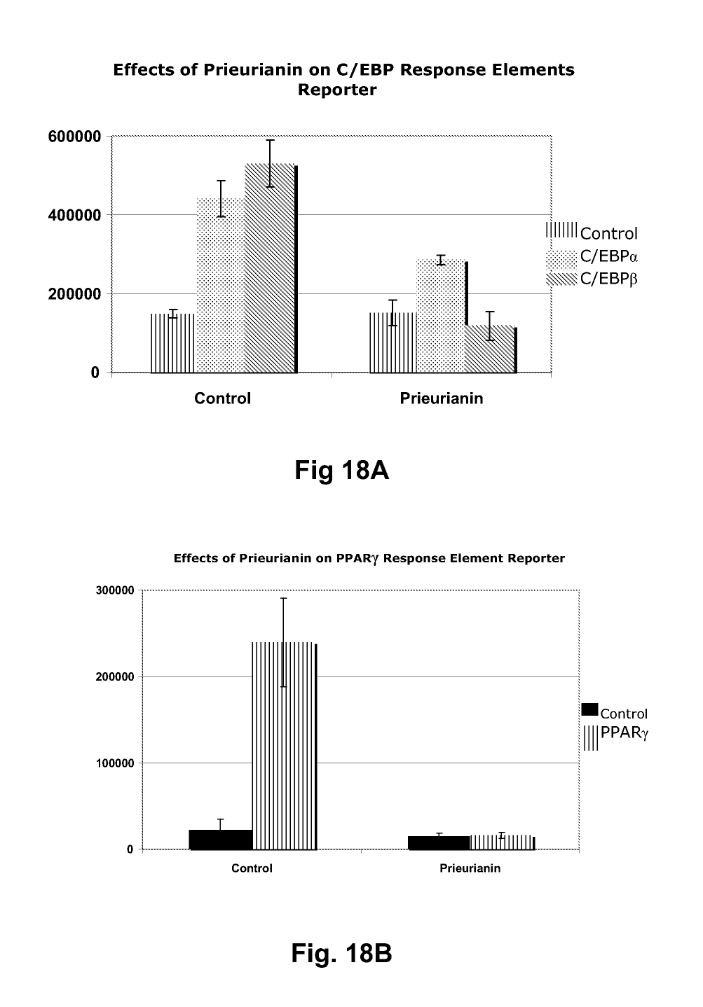

As shown in FIG. 18, prieurianin induces transactivation from the NFκB-response element mediated transcription, but inhibits the transactivation potential of C/EBPα and β, and PPARγ (see FIG. 18).

These reporter assays are thus useful to further screen for compounds that might be chemical analogs of prieurianin or its family of related small molecules that target these transcriptional processes that regulate adipogenesis. Hence, high-throughput screen for small molecules that either promotes the induction of the NFκB-mediated transcription pathway, or that cause the inhibition of transcription by C/EBPα and β, and PPARγ, that critically regulate adipogenesis, is an innovative approach for the identification of effective novel anti-obesity drugs.

### Example 13

### Effects of Bufalin and Prieurianin on Adipogenesis

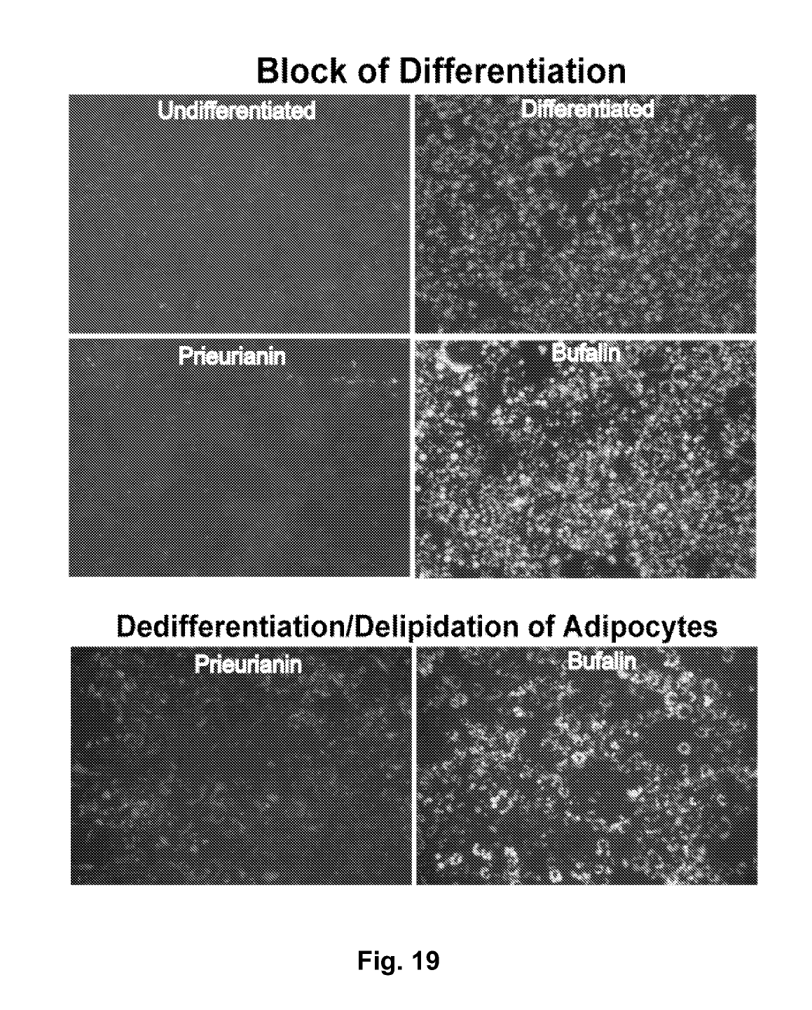

The cardiotonic steroid bufalin, a bufadienolide, stimulates the PLTP promoter like prieurianin, but did not, however, inhibit the differentiation of preadipocytes, and neither causes de-differentiation or delipidation in adipocytes (see FIG. 19) nor did it modulate the transcriptional activity of NFκB, C/EBPα and β, and PPARγ (data not shown). These results indicate the specificity of the effects of prieurianin in adipogenesis and further show that the pharmacophore of prieurianin and its derivatives can have specific actions in adipogenesis.

### Example 14

### Non-Limiting Examples of Uses and/or Indications

In another aspect, there is provided herein, a method for preventing or treating obesity in a subject, the method comprising administering to the subject a therapeutically effective amount of prieurianin. In certain embodiments, the subject is in need of such treatment or prevention.

In another aspect, there is provided herein, a method for downregulating the expression of PLTP in a subject's subcutaneous adipose tissue which comprises administering to the subject a therapeutically effective amount of prieurianin.

In another aspect, there is provided herein, a method of ameliorating or preventing adipogenesis in a mammal which comprises administering to the mammal a therapeutically effective amount of prieurianin or its derivatives.

The methods disclosed herein are also useful when the adipogenesis is associated with a disease. Also, methods can be implemented by any suitable method, including, but not limited to, administration by injection, orally or subcutaneous injection into the fat tissue.

In certain embodiments, the prevention of adipogenesis substantially decreases adipose fat tissue mass.

### Example 15

### Stimulating a NFκB Signaling Pathway In Vivo

In another aspect, there is provided herein, method for stimulating a NFκB signaling pathway in vivo to a subject in need thereof, comprising administering prieurianin to induce weight reduction and/or adiposity in the subject.

In another aspect, there is provided herein, an NFκB-response element reporter system useful for the screening of limonoids or other small molecular entity or mimicry.

In another aspect, there is provided herein a method of screening of one or more molecular entities or mimicries, comprising using an NFκB-response element reporter system.

In certain embodiments, the molecular entity or mimicry comprises one or more limonoids. Also, in certain embodiments, the limonoids are screened for efficacy in inducing weight reduction and/or adiposity.

### Example 16

### Response Elements In Vivo Through Native Promoters

In another aspect, there is provided herein, method for inhibiting one or more of C/EBPα and β, and PPARγ mediated transcriptional activation, comprising using one or more response elements in vivo through native promoters.

In another aspect, there is provided herein, method for screening for a small molecular entity for inducing weight reduction and/or adiposity, comprising inhibiting one or more of C/EBPα and β, and PPARγ mediated transcriptional activation by using one or more response elements in vivo through native promoters.

### Example 17

### Transcription Factors' Response Elements

In another aspect, there is provided herein, a method for inhibiting one or more of C/EBPα and β, and PPARγ mediated transcriptional activation, comprising using a response element driven-reporter system containing the transcription factors' response elements.

In another aspect, there is provided herein, a method for screening for a small molecular entity for inducing weight reduction and/or adiposity, comprising inhibiting one or more of C/EBPα and β, and PPARγ mediated transcriptional activation by using a response element driven-reporter system containing the transcription factors' response elements.

### Example 18

### De-Differentiation and/or Inhibiting Accumulation of Lipids in Differentiated Mature Adipocytes

In another aspect, there is provided herein, a method for causing either de-differentiation or for inhibiting accumulation of lipids in differentiated mature adipocytes, the method comprising administering an effective amount of prieurianin to the subject.

### Example 19

### Overcoming Drug-Induced Tolerance

In another aspect, there is provided herein, a method for overcoming drug-induced tolerance by administering the drug in an “on-off” or “cyclical” schedule. The method comprising: administering the drug to the subject in a specified dose for a first specified duration, refraining from administering the drug for a second specified duration, thereafter, resuming administering the drug according for one or more specified durations, and repeating the schedule as long as needed.

In certain embodiments, the method is useful for the treatment of obesity. Also, in certain embodiments, the drug comprises prieurianin.

### Example 20

### Maximal Response From the Drug Therapy

In another aspect, there is provided herein, a method that is useful for the treatment of any ailments in human in which prolong drug treatment leads to decrease efficacy, lack of response, desentization, or tolerance, in order to achieve the maximal response from the drug therapy.

In certain embodiments, the methods described herein are especially useful where the prevention of adipogenesis substantially decreases adipose fat tissue mass. Also, in particular embodiments, the methods disclosed herein are useful when the adipogenesis is a subject is associated with a disease.

In certain embodiments, the methods disclosed herein are useful when the drug delivery administration is by injection.

In certain embodiments, the methods disclosed herein are useful when the drug delivery administration, is oral.

In certain embodiments, the methods disclosed herein are useful when the drug delivery administration is by subcutaneous injection into the fat tissue.

In certain embodiments, the methods disclosed herein are useful when the drug delivery administration is dermatologically applied around areas of fat tissue.

### Example 21

### Formulating a Composition Containing Prieurianin

In another aspect, there is provided herein, a method for formulating a composition containing prieurianin or its derivatives, comprising dissolving the composition in either Cremophor or Captisol. In certain embodiments, the composition comprises prieurianin or its derivatives. Also, in certain embodiments, the composition is formulated for administering to a subject in need thereof, and wherein the composition comprises a pre-ingested form of the composition.

In a particular embodiment, the composition is formulated for administering to a subject in need thereof, and wherein the composition forms pharmaceutically active metabolites in vivo.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

### REFERENCES

1. Mercer, J. G. (2001) Dietary and genetic influences on susceptibility or resistance to weight gain on a high fat diet. Nutr Metab Cardiovasc Dis, 11, 114-7.
2. Muoio, D. M. and Newgard, C. B. (2006) Obesity-related derangements in metabolic regulation. Annu Rev Biochem, 75, 367-401.
3. Blackburn, G. L. and Waltman, B. A. (2005) Pharmacotherapy to reduce visceral fat. Clin Cornerstone, 7, 52-60.
4. Riches, F. M., Watts, G. F., Naoumova, R. P., Kelly, J. M., Croft, K. D. and Thompson, G. R. (1998) Hepatic secretion of very-low-density lipoprotein apolipoprotein B-100 studied with a stable isotope technique in men with visceral obesity. Int J Obes Relat Metab Disord, 22, 414-23.
5. Campfield, L. A., Smith, F. J., Guisez, Y., Devos, R. and Burn, P. (1995) Recombinant mouse OB protein: evidence for a peripheral signal linking adiposity and central neural networks. Science, 269, 546-9.
6. Halaas, J. L., Gajiwala, K. S., Maffei, M., Cohen, S. L., Chait, B. T., Rabinowitz, D., Lallone, R. L., Burley, S. K. and Friedman, J. M. (1995) Weight-reducing effects of the plasma protein encoded by the obese gene. Science, 269, 543-6.
7. Pelleymounter, M. A., Cullen, M. J., Baker, M. B., Hecht, R., Winters, D., Boone, T. and Collins, F. (1995) Effects of the obese gene product on body weight regulation in ob/ob mice. Science, 269, 540-3.
8. Zhang, Y., Proenca, R., Maffei, M., Barone, M., Leopold, L. and Friedman, J. M. (1994) Positional cloning of the mouse obese gene and its human homologue. Nature, 372, 425-32.
9. Kobayashi, K. (2005) Adipokines: therapeutic targets for metabolic syndrome. Curr Drug Targets, 6, 525-9.
10. Rondinone, C. M. (2006) Adipocyte-derived hormones, cytokines, and mediators. Endocrine, 29, 81-90.
11. Trayhurn, P., Bing, C. and Wood, I. S. (2006) Adipose tissue and adipokines—energy regulation from the human perspective. J Nutr, 136, 1935S-1939S.
12. Nedvidkova, J., Smitka, K., Kopsky, V. and Hainer, V. (2005) Adiponectin, an adipocyte-derived protein. Physiol Res, 54, 133-40.
13. Havel, P. J. (2000) Role of adipose tissue in body-weight regulation: mechanisms regulating leptin production and energy balance. Proc Nutr Soc, 59, 359-71.
14. Cancello, R., Tounian, A., Poitou, C. and Clement, K. (2004) Adiposity signals, genetic and body weight regulation in humans. Diabetes Metab, 30, 215-27.
15. Trayhurn, P. and Beattie, J. H. (2001) Physiological role of adipose tissue: white adipose tissue as an endocrine and secretory organ. Proc Nutr Soc, 60, 329-39.
16. Hoist, D. and Grimaldi, P. A. (2002) New factors in the regulation of adipose differentiation and metabolism. Curr Opin Lipidol, 13, 241-5.
17. Guerre-Millo, M. (2004) Adipose tissue and adipokines: for better or worse. Diabetes Metab, 30, 13-9.
18. Matsuzawa, Y., Funahashi, T., Kihara, S. and Shimomura, I. (2004) Adiponectin and metabolic syndrome. Arterioscler Thromb Vasc Biol, 24, 29-33.
19. Goldstein, B. J. and Scalia, R. (2004) Adiponectin: A novel adipokine linking adipocytes and vascular function. J Clin Endocrinol Metab, 89, 2563-8.
20. Rabin, K. R., Kamari, Y., Avni, I., Grossman, E. and Sharabi, Y. (2005) Adiponectin: linking the metabolic syndrome to its cardiovascular consequences. Expert Rev Cardiovasc Ther, 3, 465-71.
21. Maeda, K., Okubo, K., Shimomura, I., Funahashi, T., Matsuzawa, Y. and Matsubara, K. (1996) cDNA cloning and expression of a novel adipose specific collagen-like factor, apM1 (AdiPose Most abundant Gene transcript 1). Biochem Biophys Res Commun, 221, 286-9.
22. Takahashi, M., Arita, Y., Yamagata, K., Matsukawa, Y., Okutomi, K., Horie, M., Shimomura, I., Hotta, K., Kuriyama, H., Kihara, S. et al. (2000) Genomic structure and mutations in adipose-specific gene, adiponectin. Int J Obes Relat Metab Disord, 24, 861-8.
23. Arita, Y., Kihara, S., Ouchi, N., Takahashi, M., Maeda, K., Miyagawa, J., Hotta, K., Shimomura, I., Nakamura, T., Miyaoka, K. et al. (1999) Paradoxical decrease of an adipose-specific protein, adiponectin, in obesity. Biochem Biophys Res Commun, 257, 79-83.
24. Haluzik, M., Parizkova, J. and Haluzik, M. M. (2004) Adiponectin and its role in the obesity-induced insulin resistance and related complications. Physiol Res, 53, 123-9.
25. Shimada, K., Miyazaki, T. and Daida, H. (2004) Adiponectin and atherosclerotic disease. Clin Chim Acta, 344, 1-12.
26. Haluzik, M. and Haluzikova, D. (2006) The role of resistin in obesity-induced insulin resistance. Curr Opin Investig Drugs, 7, 306-11.
27. Sethi, J.K. and Vidal-Puig, A. (2005) Visfatin: the missing link between intra-abdominal obesity and diabetes? Trends Mol Med, 11, 344-7.
28. Hotamisligil, G. S. (1999) The role of TNFalpha and TNF receptors in obesity and insulin resistance. J Intern Med, 245, 621-5.
29. Cianflone, K. (1997) Acylation stimulating protein and the adipocyte. J Endocrinol, 155, 203-6.
30. Dullaart, R. P., Sluiter, W. J., Dikkeschei, L. D., Hoogenberg, K. and Van Tol, A. (1994) Effect of adiposity on plasma lipid transfer protein activities: a possible link between insulin resistance and high density lipoprotein metabolism. Eur J Clin Invest, 24, 188-94.
31. Dullaart, R. P., De Vries, R., Scheek, L., Borggreve, S. E., Van Gent, T., Dallinga-Thie, G. M., Ito, M., Nagano, M., Sluiter, W. J., Hattori, H. et al. (2004) Type 2 diabetes mellitus is associated with differential effects on plasma cholesteryl ester transfer protein and phospholipid transfer protein activities and concentrations. Scand J Clin Lab Invest, 64, 205-15.
32. Duvillard, L., Pont, F., Florentin, E., Galland-Jos, C., Gambert, P. and Verges, B. (2000) Metabolic abnormalities of apolipoprotein B-containing lipoproteins in non-insulin-dependent diabetes: a stable isotope kinetic study. Eur J Clin Invest, 30, 685-94.
33. Kaser, S., Laimer, M., Sandhofer, A., Salzmann, K., Ebenbichler, C. F. and Patsch, J. R. (2004) Effects of weight loss on PLTP activity and HDL particle size. Int J Obes Relat Metab Disord, 28, 1280-2.
34. Albers, J. J. and Cheung, M. C. (2004) Emerging roles for phospholipid transfer protein in lipid and lipoprotein metabolism. Curr Opin Lipidol, 15, 255-60.
35. Groen, A. K., Oude Elferink, R. P., Verkade, H. J. and Kuipers, F. (2004) The ins and outs of reverse cholesterol transport. Ann Med, 36, 135-45.
36. Tu, A. Y., Deeb, S. S., Iwasaki, L., Day, J. R. and Albers, J. J. (1995) Organization of human phospholipid transfer protein gene. Biochem Biophys Res Commun, 207, 552-8.
37. Whitmore, T. E., Day, J. R. and Albers, J. J. (1995) Localization of the human phospholipid transfer protein gene to chromosome 20q12-q13.1. Genomics, 28, 599-600.
38. Day, J. R., Albers, J. J., Lofton-Day, C. E., Gilbert, T. L., Ching, A. F., Grant, F. J., O'Hara, P. J., Marcovina, S. M. and Adolphson, J. L. (1994) Complete cDNA encoding human phospholipid transfer protein from human endothelial cells. J Biol Chem, 269, 9388-91.
39. Albers, J. J., Tu, A. Y., Wolfbauer, G., Cheung, M. C. and Marcovina, S. M. (1996) Molecular biology of phospholipid transfer protein. Curr Opin Lipidol, 7, 88-93.
40. Dusserre, E., Moulin, P. and Vidal, H. (2000) Differences in mRNA expression of the proteins secreted by the adipocytes in human subcutaneous and visceral adipose tissues. Biochim Biophys Acta, 1500, 8896.
41. Linder, K., Arner, P., Flores-Morales, A., Tollet-Egnell, P. and Norstedt, G. (2004) Differentially expressed genes in visceral or subcutaneous adipose tissue of obese men and women. J Lipid Res, 45, 148-54.
42. Bingle, C. D. and Craven, C. J. (2004) Meet the relatives: a family of BPI- and LBP-related proteins. Trends Immunol, 25, 53-5.
43. Beamer, L. J., Carroll, S. F. and Eisenberg, D. (1997) Crystal structure of human BPI and two bound phospholipids at 2.4 angstrom resolution. Science, 276, 1861-4.
44. Huuskonen, J., Wohlfahrt, G., Jauhiainen, M., Ehnholm, C., Teleman, O. and Olkkonen, V. M. (1999) Structure and phospholipid transfer activity of human PLTP: analysis by molecular modeling and site directed mutagenesis. J Lipid Res, 40, 1123-30.
45. Ponsin, G., Qu, S. J., Fan, H. Z. and Pownall, H. J. (2003) Structural and functional determinants of human plasma phospholipid transfer protein activity as revealed by site-directed mutagenesis of charged amino acids. Biochemistry, 42, 4444-51.
46. Tall, A. R., Krumholz, S., Olivecrona, T. and Deckelbaum, R. J. (1985) Plasma phospholipid transfer protein enhances transfer and exchange of phospholipids between very low density lipoproteins and high density lipoproteins during lipolysis. J Lipid Res, 26, 842-51.
47. Havel, R. J., Kane, J. P. and Kashyap, M. L. (1973) Interchange of apolipoproteins between chylomicrons and high density lipoproteins during alimentary lipemia in man. J Clin Invest, 52, 32-8.
48. Albers, J. J., Tollefson, J. H., Chen, C. H. and Steinmetz, A. (1984) Isolation and characterization of human plasma lipid transfer proteins. Arteriosclerosis, 4, 49-58.
49. Huuskonen, J., Olkkonen, V. M., Jauhiainen, M., Metso, J., Somerharju, P. and Ehnholm, C. (1996) Acyl chain and headgroup specificity of human plasma phospholipid transfer protein. Biochim Biophys Acta, 1303, 207-14.
50. Rao, R., Albers, J. J., Wolfbauer, G. and Pownall, H. J. (1997) Molecular and macromolecular specificity of human plasma phospholipid transfer protein. Biochemistry, 36, 3645-53.
51. Nishida, H. I. and Nishida, T. (1997) Phospholipid transfer protein mediates transfer of not only phosphatidylcholine but also cholesterol from phosphatidylcholine-cholesterol vesicles to high density lipoproteins. J Biol Chem, 272, 6959-64.
52. Tu, A. Y., Nishida, H. I. and Nishida, T. (1993) High density lipoprotein conversion mediated by human plasma phospholipid transfer protein. J Biol Chem, 268, 23098-105.
53. Jauhiainen, M., Metso, J., Pahlman, R., Blomqvist, S., van Tol, A. and Ehnholm, C. (1993) Human plasma phospholipid transfer protein causes high density lipoprotein conversion. J Biol Chem, 268, 4032-6.
54. Pussinen, P., Jauhianinen, M., Metso, J., Tyynela, J. and Ehnholm, C. (1995) Pig plasma phospholipid transfer protein facilitates HDL interconversion. J Lipid Res, 36, 975-85.
55. Jiang, X. C., Bruce, C., Mar, J., Lin, M., Ji, Y., Francone, O. L. and Tall, A. R. (1999) Targeted mutation of plasma phospholipid transfer protein gene markedly reduces high-density lipoprotein levels. J Clin Invest, 103, 907-14.
56. Foger, B., Santamarina-Fojo, S., Shamburek, R. D., Parrot, C. L., Talley, G. D. and Brewer, H. B., Jr. (1997) Plasma phospholipid transfer protein. Adenovirus-mediated overexpression in mice leads to decreased plasma high density lipoprotein (HDL) and enhanced hepatic uptake of phospholipids and cholesteryl esters from HDL. J Biol Chem, 272, 27393-400.
57. Ehnholm, S., van Dijk, K. W., van 't Hof, B., van der Zee, A., Olkkonen, V. M., Jauhiainen, M., Hofker, M., Havekes, L. and Ehnholm, C. (1998) Adenovirus mediated overexpression of human phospholipid transfer protein alters plasma HDL levels in mice. J Lipid Res, 39, 1248-53.
58. Yamashita, S., Sakai, N., Hirano, K., Ishigami, M., Maruyama, T., Nakajima, N. and Matsuzawa, Y. (2001) Roles of plasma lipid transfer proteins in reverse cholesterol transport. Front Biosci, 6, D366-87.
59. Jiang, X., Francone, O.L., Bruce, C., Milne, R., Mar, J., Walsh, A., Breslow, J. L. and Tall, A. R. (1996) Increased prebeta-high density lipoprotein, apolipoprotein AI, and phospholipid in mice expressing the human phospholipid transfer protein and human apolipoprotein AI transgenes. J Clin Invest, 98, 237380.
60. van Haperen, R., van Tol, A., Vermeulen, P., Jauhiainen, M., van Gent, T., van den Berg, P., Ehnholm, S., Grosveld, F., van der Kamp, A. and de Crom, R. (2000) Human plasma phospholipid transfer protein increases the antiatherogenic potential of high density lipoproteins in transgenic mice. Arterioscler Thromb Vasc Biol, 20, 1082-8.
61. Dullaart, R. P. and van Tol, A. (2001) Role of phospholipid transfer protein and prebeta-high density lipoproteins in maintaining cholesterol efflux from Fu5AH cells to plasma from insulin-resistant subjects. Scand J Clin Lab Invest, 61, 69-74.
62. Jiang, X. C., Qin, S., Qiao, C., Kawano, K., Lin, M., Skold, A., Xiao, X. and Tall, A. R. (2001) Apolipoprotein B secretion and atherosclerosis are decreased in mice with phospholipid-transfer protein deficiency. Nat Med, 7, 847-52.
63. Albers, J. J., Tu, A. Y., Paigen, B., Chen, H., Cheung, M. C. and Marcovina, S. M. (1996) Transgenic mice expressing human phospholipid transfer protein have increased HDL/non-HDL cholesterol ratio. Int J Clin Lab Res, 26, 262-7.
64. Schlitt, A., Bickel, C., Thumma, P., Blankenberg, S., Rupprecht, H. J., Meyer, J. and Jiang, X. C. (2003) High plasma phospholipid transfer protein levels as a risk factor for coronary artery disease. Arterioscler Thromb Vasc Biol, 23, 1857-62.
65. Lay, J., de Crom, R., van Gent, T., van Haperen, R., Scheek, L., Sadeghi-Niaraki, F. and van Tol, A. (2004) Elevation of plasma phospholipid transfer protein increases the risk of atherosclerosis despite lower apolipoprotein B-containing lipoproteins. J Lipid Res, 45, 805-11.
66. Yang, X.P., Yan, D., Qiao, C., Liu, R. J., Chen, J. G., Li, J., Schneider, M., Lagrost, L., Xiao, X. and Jiang, X. C. (2003) Increased atherosclerotic lesions in apoE mice with plasma phospholipid transfer protein overexpression. Arterioscler Thromb Vasc Biol, 23, 1601-7.
67. Lie, J., Moerland, M., van Gent, T., van Haperen, R., Scheek, L., Sadeghi-Niaraki, F., de Crom, R. and van Tol, A. (2006) Sex differences in atherosclerosis in mice with elevated phospholipid transfer protein activity are related to decreased plasma high density lipoproteins and not to increased production of triglycerides. Biochim Biophys Acta, 1761, 1070-7.
68. van Haperen, R., van Tol, A., van Gent, T., Scheek, L., Visser, P., van der Kamp, A., Grosveld, F. and de Crom, R. (2002) Increased risk of atherosclerosis by elevated plasma levels of phospholipid transfer protein. J Biol Chem, 277, 48938-43.
69. van Tol, A. (2002) Phospholipid transfer protein. Curr Opin Lipidol, 13, 135-9.
70. Murdoch, S. J., Carr, M. C., Hokanson, J. E., Brunzell, J. D. and Albers, J. J. (2000) PLTP activity in premenopausal women. Relationship with lipoprotein lipase, HDL, LDL, body fat, and insulin resistance. J Lipid Res, 41, 237-44.
71. Kaser, S., Sandhofer, A., Foger, B., Ebenbichler, C. F., Igelseder, B., Malaimare, L., Paulweber, B. and Patsch, J. R. (2001) Influence of obesity and insulin sensitivity on phospholipid transfer protein activity. Diabetologia, 44, 1111-7.
72. Cheung, M. C., Knopp, R. H., Retzlaff, B., Kennedy, H., Wolfbauer, G. and Albers, J. J. (2002) Association of plasma phospholipid transfer protein activity with IDL and buoyant LDL: impact of gender and adiposity. Biochim Biophys Acta, 1587, 53-9.
73. Murdoch, S. J., Kahn, S. E., Albers, J. J., Brunzell, J. D. and Purnell, J. Q. (2003) PLTP activity decreases with weight loss: changes in PLTP are associated with changes in subcutaneous fat and FFA but not IAF or insulin sensitivity. J Lipid Res, 44, 1705-12.
74. Lee, J. H., Reed, D. R., Li, W. D., Xu, W., Joo, E. J., Kilker, R. L., Nanthakumar, E., North, M., Sakul, H., Bell, C. et al. (1999) Genome scan for human obesity and linkage to markers in 20q13. Am J Hum Genet, 64, 196-209.
75. Norman, R. A., Tataranni, P. A., Pratley, R., Thompson, D. B., Hanson, R. L., Prochazka, M., Baier, L., Ehm, M. G., Sakul, H., Foroud, T. et al. (1998) Autosomal genomic scan for loci linked to obesity and energy metabolism in Pima Indians. Am J Hum Genet, 62, 659-68.
76. Lembertas, A. V., Perusse, L., Chagnon, Y. C., Fisler, J. S., Warden, C. H., Purcell-Huynh, D. A., Dionne, F. T., Gagnon, J., Nadeau, A., Lusis, A. J. et al. (1997) Identification of an obesity quantitative trait locus on mouse chromosome 2 and evidence of linkage to body fat and insulin on the human homologous region 20q. J Clin Invest, 100, 1240-7.
77. Pomp, D. (1997) Genetic dissection of obesity in polygenic animal models. Behav Genet, 27, 285-306.
78. Mehrabian, M., Wen, P. Z., Fisler, J., Davis, R. C. and Lusis, A. J. (1998) Genetic loci controlling body fat, lipoprotein metabolism, and insulin levels in a multifactorial mouse model. J Clin Invest, 101, 248596.
79. Ji, J., Watts, G. F., Johnson, A. G., Chan, D. C., Ooi, E. M., Rye, K. A., Serone, A. P. and Barrett, P. H. (2006) High-density lipoprotein (HDL) transport in the metabolic syndrome: application of a new model for HDL particle kinetics. J Clin Endocrinol Metab, 91, 973-9.
80. Ashrafi, K., Chang, F. Y., Watts, J. L., Fraser, A. G., Kamath, R. S., Ahringer, J. and Ruvkun, G. (2003) Genome-wide RNAi analysis of Caenorhabditis elegans fat regulatory genes. Nature, 421, 268-72.
81. Kudoh, K., Ramanna, M., Ravatn, R., Elkahloun, A. G., Bittner, M. L., Meltzer, P. S., Trent, J. M., Dalton, W. S. and Chin, K. V. (2000) Monitoring the expression profiles of doxorubicin-induced and doxorubicin-resistant cancer cells by cDNA microarray. Cancer Res, 60, 4161-6.
82. Bruce, C., Chouinard, R. A., Jr. and Tall, A. R. (1998) Plasma lipid transfer proteins, high-density lipoproteins, and reverse cholesterol transport. Annu Rev Nutr, 18, 297-330.
83. Sarker, S. D., Savchenko, T., Whiting, P., Sik, V. and Dinan, L. (1997) Two limonoids from Turraea obtusifolia (Meliaceae), prieurianin and rohitukin, antagonise 20-hydroxyecdysone action in a Drosophila cell line. Arch Insect Biochem Physiol, 35, 211-7.
84. Koul, O., Daniewski, W. M., Multani, J. S., Gumulka, M. and Singh, G. (2003) Antifeedant effects of the limonoids from Entandrophragma candolei (Meliaceae) on the gram pod borer, Helicoverpa armigera (Lepidoptera: Noctuidae). J Agric Food Chem, 51, 7271-5.
85. Bray, G. A. and Bellanger, T. (2006) Epidemiology, trends, and morbidities of obesity and the metabolic syndrome. Endocrine, 29, 109-17.
86. Pollex, R. L. and Hegele, R. A. (2006) Genetic determinants of the metabolic syndrome. Nat Clin Pract Cardiovasc Med, 3, 482-9.
87. Dubuc, P. U. (1976) The development of obesity, hyperinsulinemia, and hyperglycemia in ob/ob mice. Metabolism, 25, 1567-74.
88. Simons, P. J., van den Pangaart, P. S., van Roomen, C. P., Aerts, J. M. and Boon, L. (2005) Cytokinemediated modulation of leptin and adiponectin secretion during in vitro adipogenesis: evidence that tumor necrosis factor-alpha- and interleukin-1beta-treated human preadipocytes are potent leptin producers. Cytokine, 32, 94-103.
89. Swannie, H. C. and Kaye, S. B. (2002) Protein kinase C inhibitors. Curr Oncol Rep, 4, 37-46.
90. Harp, J. B. (2004) New insights into inhibitors of adipogenesis. Curr Opin Lipidol, 15, 303-7.
91. Ailhaud, G. (2006) Adipose tissue as a secretory organ: from adipogenesis to the metabolic syndrome. C R Biol, 329, 570-7; discussion 653-5.
92. Erickson, J. C., Hollopeter, G. and Palmiter, R. D. (1996) Attenuation of the obesity syndrome of ob/ob mice by the loss of neuropeptide Y. Science, 274, 1704-7.
93. Birk, R. Z. and Rubinstein, M. (2006) IFN-alpha induces apoptosis of adipose tissue cells. Biochem Biophys Res Commun, 345, 669-74.
94. Koopman, G., Reutelingsperger, C. P., Kuijten, G. A., Keehnen, R. M., Pals, S. T. and van Oers, M. H. (1994) Annexin V for flow cytometric detection of phosphatidylserine expression on B cells undergoing apoptosis. Blood, 84, 1415-20.
95. Gullicksen, P. S., Hausman, D. B., Dean, R. G., Hartzell, D. L. and Baile, C. A. (2003) Adipose tissue cellularity and apoptosis after intracerebroventricular injections of leptin and 21 days of recovery in rats. Int J Obes Relat Metab Disord, 27, 302-12.
96. Hirsch, J. and Gallian, E. (1968) Methods for the determination of adipose cell size in man and animals. J Lipid Res, 9, 110-9.
97. Korsmeyer, S. J., Shutter, J. R., Veis, D. J., Merry, D. E. and Oltvai, Z. N. (1993) Bcl-2/Bax: a rheostat that regulates an anti-oxidant pathway and cell death. Semin Cancer Biol, 4, 327-32.
98. Kaser, S., Foger, B., Ebenbichler, C. F., Kirchmair, R., Gander, R., Ritsch, A., Sandhofer, A. and Patsch, J. R. (2001) Influence of leptin and insulin on lipid transfer proteins in human hepatoma cell line, HepG2. Int J Obes Relat Metab Disord, 25, 1633-9.
99. Shillabeer, G., Forden, J. M., Russell, J. C. and Lau, D. C. (1990) Paradoxically slow preadipocyte replication and differentiation in corpulent rats. Am J Physiol, 258, E368-76.
100. Yan, H., Aziz, E., Shillabeer, G., Wong, A., Shanghavi, D., Kermouni, A., Abdel-Hafez, M. and Lau, D. C. (2002) Nitric oxide promotes differentiation of rat white preadipocytes in culture. J Lipid Res, 43, 2123-9.
101. Ntambi, J. M. and Young-Cheul, K. (2000) Adipocyte differentiation and gene expression. J Nutr, 130, 3122S-3126S.
102. Negrel, R., Grimaldi, P. and Ailhaud, G. (1978) Establishment of preadipocyte clonal line from epididymal fat pad of ob/ob mouse that responds to insulin and to lipolytic hormones. Proc Natl Acad Sci USA, 75, 6054-8.
103. Gurley, L. R., Umbarger, K. O., Kim, J. M., Bradbury, E. M. and Lehnert, B. E. (1998) High-performance liquid chromatographic analysis of staurosporine in vivo. Its translocation and pharmacokinetics in rats. J Chromatogr B Biomed Sci Appl, 712, 211-24.
104. Fuse, E., Kuwabara, T., Sparreboom, A., Sausville, E. A. and Figg, W. D. (2005) Review of UCN-01 development: a lesson in the importance of clinical pharmacology. J Clin Pharmacol, 45, 394-403.
105. Miard, S. and Fajas, L. (2005) Atypical transcriptional regulators and cofactors of PPARgamma. Int J Obes (Lond), 29 Suppl 1, S10-2.
106. Qiao, L., Schaack, J. and Shao, J. (2006) Suppression of adiponectin gene expression by histone deacetylase inhibitor valproic acid. Endocrinology, 147, 865-74.
107. Shinohara, O., Murata, Y. and Shimizu, M. (1992) Endothelin-1 suppression of rat adipocyte precursor cell differentiation in serum-free culture. Endocrinology, 130, 2031-6.
108. Shinohara, O., Murata, Y., Kubota, C. and Shinagawa, T. (1994) Protein kinase C inhibitors enhance differentiation of rat adipocyte precursor cells in serum-free culture. Biochem Med Metab Biol, 51, 8590.
109. Wright, H. M., Clish, C. B., Mikami, T., Hauser, S., Yanagi, K., Hiramatsu, R., Serhan, C. N. and Spiegelman, B. M. (2000) A synthetic antagonist for the peroxisome proliferator-activated receptor gamma inhibits adipocyte differentiation. J Biol Chem, 275, 1873-7.

1-48. (canceled)

49. A dual mechanistic anorexigenic and anti-adipogenic anti-obesity agent, comprising a limonoid or a derivative thereof.

50. The agent of claim 49, wherein the limonoid comprises prieurianin or a derivative thereof.

51. A body weight reducing composition for use in a subject in need thereof, comprising an effective amount of a limonoid or a derivative thereof.

52. The composition of claim 51, wherein the limonoid comprises prieurianin or a derivative thereof.

53. A method for treating a subject, comprising administering an effective amount of a limonoid, or a derivative thereof, to the subject.

54. The composition of claim 53, wherein the limonoid comprises prieurianin or a derivative thereof.

55. The method of claim 53, wherein the composition is administered for one or more of:
inducing weight loss and/or reduction in food intake;
decreasing visceral and subcutaneous adipose tissues;
decreasing serum non-esterified fatty acid levels;
inhibiting/preventing the proliferation and/or differentiation of preadipocytes into mature adipocytes;
causing de-differentiation, and/or a loss of fat accumulation in adipocytes;
inhibiting accumulation of lipids/fat in differentiated mature adipocytes;
ameliorating or preventing adipogenesis; and
preventing or treating obesity.

56. The method of claim 55, wherein the limonoid comprises prieurianin or a derivative thereof.

57. The method of claim 55, wherein the subject comprises a mammal considered obese.

58. The method of claim 55, wherein a therapeutically effective amount of the limonoid prieurianin or its derivatives is delivered by injection or any pharmacologically administrable route.

59. The method of claim 55, wherein a therapeutically effective amount of prieurianin or its derivatives is delivered by subcutaneous injection into the fat tissue.

60. The method of claim 55, wherein a therapeutically effective amount of prieurianin or its derivatives is delivered by applying around areas of fat tissue.

61. A method for screening for a small molecular entity for inducing reduction in weight and/or adiposity in a subject in need thereof, the method comprising the step of:
inhibiting one or more of C/EBPα and β, and PPARγ mediated transcriptional activation in the subject by using one or more response elements in vivo through native promoters.

62. A method for inhibiting one or more of C/EBPα and β, and PPARγ mediated transcriptional activation in a subject in need thereof, the method comprising the step of: using a response element driven-reporter system containing the transcription factors' response elements.

63. A method for screening for a small molecular entity for inducing reduction in weight and/or adiposity in a subject in need thereof, the method comprising the step of:
inhibiting one or more of C/EBPα and β, and PPARγ mediated transcriptional activation by using a response element driven-reporter system containing the transcription factors' response elements.

64. A method for causing either de-differentiation or for inhibiting accumulation of lipids in differentiated mature adipocytes in a subject in need thereof, the method comprising the step of:
administering an effective amount of prieurianin to the subject.

65. A method for overcoming drug-induced tolerance by administering the drug in an “on-off” or “cyclical” schedule, the method comprising:
administering the drug to the subject in a specified dose for a first specified duration,
refraining from administering the drug for a second specified duration,
thereafter, resuming administering the drug according for one or more specified durations, and, optionally
repeating the schedule.

66. The method of claim 65, wherein the method is used for the treatment of obesity.

67. The method of claim 65, wherein in the drug comprises a limonoid or a derivative thereof.

68. The method of claim 65, wherein in the drug comprises prieurianin or a derivative thereof.

69. The method of claim 65, wherein the method is used for the treatment of humans in which prolong drug treatment leads to decrease efficacy, lack of response, desentization, or tolerance, in order to achieve the maximal response from the drug therapy.

70. A method for formulating a composition containing prieurianin or its derivatives, the method comprising the step of:
dissolving the composition in a pharmacologically active composition that can be administered to a subject.

71. The composition of claim 70, wherein the composition is formulated for administering to a subject in need thereof, and wherein the composition forms a pharmaceutically active metabolites in vivo.

72. A method for activating phospholipid transfer protein (PLTP) activity in a subject in need thereof, the method comprising the step of administering an effective amount of a limonoid or a derivative thereof.

73. The method of claim 72, wherein the limonoid comprises prieurianin or a derivative thereof.
